(19) United States
(12) Patent Application Publication
(10) Pub. No.: US 2021/0277363 A1
(43) Pub. Date: Sep. 9, 2021

(54) Manufacture of Virus

(71) Applicant: Western Oncolytics Ltd., Pittsburgh, PA (US)

(72) Inventors: Paul Andrew WRIGHT, San Diego, CA (US); Katy WACK, Pittsburgh, PA (US); Mingrui ZHANG, Pittsburgh, PA (US); Jacqueline VASSEUR, Pittsburgh, PA (US); Susan Nicole HOLLEY, San Diego, CA (US); August Allen RENSHAW, San Diego, CA (US); Guangyu ZHU, San Diego, CA (US); Michelle Catherine VENDEL, San Diego, CA (US)

(21) Appl. No.: 17/324,756

(22) Filed: May 19, 2021

Related U.S. Application Data
(63) Continuation of application No. PCT/US2019/062643, filed on Nov. 21, 2019.
(60) Provisional application No. 62/770,577, filed on Nov. 21, 2018.

(51) Int. Cl. C12N 7/00

(52) U.S. Cl. CPC C12N 7/00; C12N 2710/24151

(57) ABSTRACT

Provided herein are methods of manufacturing viruses. Also provided herein are bioreactors comprising virus-infected host cells comprising viruses. In some aspects, the present disclosure relates to production of recombinant oncolytic viruses, e.g., recombinant vaccinia viruses.

### CROSS-REFERENCE

This application is a bypass continuation of PCT/US2019/062643, filed Nov. 21, 2019, which claims the benefit of U.S. Provisional Application No. 62/770,577 filed Nov. 21, 2018, which is incorporated by reference herein in its entirety.

### BACKGROUND

Viruses can find use in many areas, e.g., therapeutics, e.g., oncolytic therapeutics. Oncolytic viruses hold great promise for the treatment of many types of cancer. In many instances, recombinant oncolytic viruses generated through genetic engineering of naturally occurring viruses prove to be effective tools for eradicating cancer cells. However, therapeutic applications of oncolytic viruses have requirements of high standard in many aspects. For example, clinical applicable viruses may need to be of high purity. Also high titer of viruses may be needed in order to be therapeutically effective. In addition, how to scale up viral production for therapeutic applications also remains a great challenge. Therefore, an improved method of manufacturing viruses is desirable.

### SUMMARY

In one aspect, the present disclosure provides a method of manufacture, comprising: growing a culture comprising a plurality of virus infected host cells in a bioreactor to produce at least about 50 plaque forming units (PFU) to about 350 PFU of a recombinant oncolytic virus per host cell, wherein said bioreactor comprises a surface area of about 0.2 m2 to about 500 m2.

In one aspect, the present disclosure provides a method of manufacture, comprising: (i) growing a culture comprising a plurality of virus infected host cells in a bioreactor; and (ii) harvesting from said culture a population of recombinant oncolytic virus, wherein said harvesting comprises lysing said culture to produce a lysate comprising said population of recombinant oncolytic virus, and (iii) clarifying said lysate to produce a population of purified recombinant oncolytic virus, wherein said population of purified recombinant oncolytic virus comprises at least about 50% to about 90% of said population recombinant oncolytic virus in said lysate, and wherein said bioreactor comprises a surface area of about 0.2 m2 to about 500 m2.

In one aspect, the present disclosure provides a method of manufacture, comprising: harvesting from a culture comprising a plurality of virus infected host cells, grown in a bioreactor, a population of a recombinant oncolytic virus, wherein said population comprises about 1.5×10¹¹ to about 5×10¹³ PFU of said recombinant oncolytic virus, and wherein said bioreactor comprises a surface area of about 0.2 m2 to about 500 m2.

In one aspect, the present disclosure provides a method of manufacture, comprising: harvesting from a culture comprising a plurality of virus infected host cells, grown in a bioreactor, a population of a recombinant oncolytic virus, wherein said population comprises a viral titer of about 1.5×10⁸ to about 2×10¹⁰ PFU per mL of said culture, wherein bioreactor comprises a surface area of about 0.2 m2 to about 500 m2.

In one aspect, the present disclosure provides a method of manufacture, comprising: (i) growing a culture comprising a plurality of virus infected host cells, in a bioreactor; and (ii) harvesting from said culture a population of recombinant oncolytic virus, wherein said bioreactor comprises a surface area of about 0.2 m2 to about 500 m2, and wherein said harvesting is carried out in an alkaline condition.

In one aspect, the present disclosure provides a method of manufacture, comprising growing a culture comprising a plurality of virus infected host cells in a bioreactor to produce a population of recombinant oncolytic virus, wherein said plurality of virus infected host cells have been infected with a virus at a multiplicity of infection (m.o.i.) of about 0.0001 to about 0.01 PFU/cell, and wherein said bioreactor comprises a surface area of about 0.2 m2 to about 500 m2.

In one aspect, the present disclosure provides a method of manufacture, comprising: (i) growing a culture comprising a plurality of virus infected host cells in a bioreactor, wherein said plurality of virus infected host cells have been infected with a virus at a multiplicity of infection (m.o.i.) of about 0.0001 to about 0.01, and (ii) harvesting from said culture a population of a recombinant oncolytic virus, wherein said population comprises about 1.5×10¹¹ to about 5×10¹³ PFU of said recombinant oncolytic virus.

In some embodiments, said bioreactor comprises a fixed-bed. In some embodiments, said fixed-bed comprises microcarriers. In some embodiments, said fixed bed comprises a volume of about 0.01 L to about 25 L. In some embodiments, said fixed bed comprises a compaction density of at least about 80 g/L to about 144 g/L. In some embodiments, said fixed bed comprises a compaction density of at least about 96 g/L.

In some embodiments, said plurality of virus infected host cells are seeded at a density of at least about 1,000 cells/cm2 to about 150,000 cells/cm2 in said bioreactor. In some embodiments, said culture comprising said virus-infected host cells is grown for about 24 hours to about 96 hours. In some embodiments, said culture comprising said virus-infected host cells is grown for about 48 hours to about 72 hours. In some embodiments, said culture comprising said virus-infected host cells comprises a dissolved oxygen tension (DOT) level of at least about 20% to about 100%. In some embodiments, said culture comprising said virus-infected host cells is maintained at a temperature of about 32° C. to about 38° C. In some embodiments, said culture comprising said virus-infected host cells is maintained at a pH of about 7.0 to about 7.5. In some embodiments, said plurality of virus infected cells have been infected with a virus at a multiplicity of infection (m.o.i.) of about 0.0005 to about 0.2.

In some embodiments, the method further comprises lysing said plurality of virus infected host cells by incubating in an alkaline buffer. In some embodiments, the method further comprises lysing said plurality of virus infected host cells by incubating with an enzyme to degrade cell debris. In some embodiments, the method further comprises lysing said plurality of virus infected host cells by incubating with an enzyme to degrade cell debris and an alkaline buffer. In some embodiments, said harvesting comprises lysing said plurality of virus infected host cells by incubating in an alkaline buffer. In some embodiments, said harvesting comprises lysing said plurality of virus infected host cells by incubating with an enzyme to degrade cell debris. In some embodiments, said harvesting comprises lysing said plurality of virus infected host cells by incubating with an enzyme to degrade cell debris and an alkaline buffer. In some embodiments, a concentration of said enzyme, during said incubating, is from about 50 IU/mL to about 200 IU/mL. In some embodiments, said incubating is for about 2 hours to about 8 hours. In some embodiments, said incubating is at a temperature of about 22° C. to about 28° C. In some embodiments, said enzyme to degrade cell debris comprises Benzonase®. In some embodiments, said alkaline buffer comprises a pH of above 8. In some embodiments, said alkaline buffer comprises a pH of above 8.0 to about 10.0. In some embodiments, said alkaline buffer comprises a pH of about 9.5. In some embodiments, said alkaline buffer comprises a Tris buffer. In some embodiments, the method further comprises monitoring pH during said incubating.

In some embodiments, the method further comprises conducting a filtration to generate a filtered lysate. In some embodiments, the method further comprises treating said filtered lysate with an enzyme to degrade cell debris. In some embodiments, said treating is in an alkaline condition. In some embodiments, said treating is for about 10 hours to about 24 hours. In some embodiments, said treating is followed by a tangential flow filtration of said filtered lysate to generate a purified preparation of said recombinant oncolytic virus. In some embodiments, said tangential flow filtration comprises concentrating said filtered lysate followed by at least a first and a second round of diafiltration. In some embodiments, said first round of diafiltration comprises a buffer exchange with a buffer comprising a pH of about 7.0 to about 7.5. In some embodiments, said first round of diafiltration comprises about 10 volumes of said buffer exchange. In some embodiments, said buffer comprising a pH of about 7.0 to about 7.5 comprises a Tris concentration of about 15 mM to about 40 mM. In some embodiments, said buffer comprising a pH of about 7.0 to about 7.5 further comprises at least about 5% to at least about 20% sucrose, by volume. In some embodiments, said second round of diafiltration comprises a buffer exchange with a buffer comprising a pH of above 7.5. In some embodiments, said second round of diafiltration comprises about 6 volumes of said buffer exchange with said buffer comprising a pH of above 7.5. In some embodiments, said buffer comprising a pH of above 7.5 comprises a Tris concentration of about 10 mM to about 30 mM. In some embodiments, said buffer comprising a pH of above 7.5 further comprises at least about 5% to at least about 10% sucrose, by volume.

In some embodiments, said purified preparation of said recombinant oncolytic virus comprises from about 5 ng to about 100 ng of host cell DNA in a unit dose of said recombinant oncolytic virus. In some embodiments, said purified preparation of said recombinant oncolytic virus comprises from about 0.1 μg to about 10 μg of host cell protein in a unit dose of said recombinant oncolytic virus. In some embodiments, said unit dose comprises about 1×10⁹ to about 1×10¹³ PFU of said recombinant oncolytic virus. In some embodiments, said purified preparation of said recombinant oncolytic virus comprises up to about 400 ng, about 200 ng, about 100 ng, about 50 ng, or about 40 ng of host cell DNA per 5×10⁹ PFU of said recombinant oncolytic virus. In some embodiments, said purified preparation of said recombinant oncolytic virus comprises up to about 80 ng, about 40 ng, about 20 ng, about 10 ng, or about 8 ng of host cell DNA per 1×10⁹ PFU of said recombinant oncolytic virus. In some embodiments, said purified preparation of said recombinant oncolytic virus comprises up to about 7.5 μg, about 7 μg, about 6 μg, about 5 μg, or about 4 μg of host cell protein per 5×10⁹ PFU of said recombinant oncolytic virus. In some embodiments, said purified preparation of said recombinant oncolytic virus comprises up to about 1.5 μg, about 1.4 μg, about 1.2 μg, about 1 μg, or about 0.8 μg of host cell protein per 1×10⁹ PFU of said recombinant oncolytic virus. In some embodiments, said purified preparation of said recombinant oncolytic virus comprises up to about 100 ng, about 60 ng, about 50 ng, about 30 ng, or about 25 ng of serum albumin that is used in said culture per 5×10⁹ PFU of said recombinant oncolytic virus.

In some embodiments, said host cell is selected from a group consisting of a HeLa cell, 293 cells, and Vero cells. In some embodiments, said recombinant oncolytic virus comprises a recombinant oncolytic vaccinia virus.

In one aspect, the present disclosure provides a composition comprising a population of recombinant oncolytic virus produced by the method as described herein.

In one aspect, the present disclosure provides a bioreactor comprising a culture of host cells comprising at least about 50 PFU to about 350 PFU of a recombinant oncolytic virus per host cell.

In some embodiments, the bioreactor comprises a fixed bed, wherein said fixed bed comprises said culture of host cells. In some embodiments, said culture of host cells is propagated in microcarriers within said fixed bed. In some embodiments, the bioreactor comprises said culture of host cells, wherein said culture comprises a host cell density of about 1,000 cells/cm2 to about 10,000 cells/cm2 of said fixed bed. In some embodiments, said fixed bed comprises a volume of about 0.01 L to about 25 L. In some embodiments, said fixed bed comprises a compaction density of at least about 80 g/L to about 144 g/L. In some embodiments, said fixed bed comprises a surface area of about 0.2 m2 to about 500 m2.

In one aspect, the present disclosure provides a bioreactor comprising about 1.5×10¹¹ to about 5×10¹³ PFU of a recombinant oncolytic virus. In some embodiments, the bioreactor comprises a fixed bed, wherein said fixed bed comprises a culture comprising said recombinant oncolytic virus. In some embodiments, said fixed bed comprises a volume of about 0.01 L to about 25 L. In some embodiments, said fixed bed comprises a compaction density of at least about 80 g/L to about 144 g/L. In some embodiments, said fixed bed comprises a surface area of about 0.2 m2 to about 500 m2. In some embodiments, said recombinant oncolytic virus comprises a recombinant oncolytic vaccinia virus.

In one aspect, the present disclosure provides a method for producing a recombinant oncolytic virus, the method comprising: (i) contacting a culture of host cells in a bioreactor with a vaccinia virus at a multiplicity of infection of about 0.001 PFU/cell; (ii) growing said culture for a period of about 72 hours to generate a further culture comprising a plurality of virus infected host cells; (iii) harvesting from said further culture comprising said plurality of virus infected host cells a population of a recombinant oncolytic virus, wherein said population comprises about 1.5×10¹¹ to about 5×10¹³ PFU of said recombinant oncolytic virus, wherein said bioreactor comprises a surface area of about 0.2 m2 to about 500 m2, and wherein said harvesting is carried out in an alkaline condition.

In some embodiments, said bioreactor comprises a fixed-bed. In some embodiments, said fixed-bed comprises microcarriers. In some embodiments, said fixed bed comprises a volume of about 0.01 L to about 25 L. In some embodiments, said fixed bed comprises a compaction density of at least about 80 g/L to about 144 g/L. In some embodiments, said fixed bed comprises a compaction density of at least about 96 g/L. In some embodiments, said host cells are seeded at a density of at least about 1,000 cells/cm2 to about 150,000 cells/cm2 in said bioreactor. In some embodiments, said further culture comprises a dissolved oxygen tension (DOT) level of at least about 20% to about 100%. In some embodiments, said further culture comprises a dissolved oxygen tension (DOT) level of about 50%. In some embodiments, said further culture is maintained at a temperature of about 32° C. to about 38° C. In some embodiments, said further culture is maintained at a temperature of about 36° C. In some embodiments, said further culture is maintained at a pH of about 7.0 to about 7.5. In some embodiments, said further culture is maintained at a pH of about 7.2. In some embodiments, said harvesting comprises lysing said plurality of virus infected host cells by incubating with an enzyme to degrade cell debris and an alkaline buffer. In some embodiments, a concentration of said enzyme to degrade cell debris, during said incubating, is from about 50 IU/mL to about 200 IU/mL. In some embodiments, said incubating is at a temperature of about 22° C. to about 28° C. In some embodiments, said incubating is at a temperature of about 20° C., 21° C., 22° C., 23° C., 24° C., 25° C., 26° C., 27° C., 28° C., 29° C., or up to about 30° C. In some embodiments, said incubating is at a temperature of about 25° C. In some embodiments, said incubating is at a temperature of about 27° C. In some embodiments, said enzyme to degrade cell debris comprises Benzonase®. In some embodiments, said cell enzyme to degrade cell debris comprises Benzonase® and wherein said incubating is in the presence of said Benzonase® at a concentration of about 150 IU/mL, at a temperature of about 27° C., for a period of about 5 hours. In some embodiments, said alkaline buffer comprises a pH of above 8. In some embodiments, said alkaline buffer comprises a pH of above 8.0 to about 10.0. In some embodiments, said alkaline buffer comprises a pH of about 9.5. In some embodiments, said alkaline buffer comprises a Tris buffer.

In some embodiments, the method further comprises monitoring pH during said incubating.

In some embodiments, the method further comprises conducting a filtration to generate a filtered lysate. In some embodiments, the method further comprises treating said filtered lysate with a cell lysing enzyme. In some embodiments, said treating is in an alkaline condition. In some embodiments, said treating is for about 10 hours to about 24 hours. In some embodiments, said treating is for about 18 hours at a temperature of about 4° C. In some embodiments, said treating is followed by a tangential flow filtration of said filtered lysate to generate a purified preparation of said recombinant oncolytic virus. In some embodiments, said tangential flow filtration comprises concentrating said filtered lysate followed by at least a first and a second round of diafiltration. In some embodiments a second round of concentration can be performed. In some embodiments, said first round of diafiltration comprises a buffer exchange with a buffer comprising a pH of about 9.0 to about 9.5. In some embodiments, said first round of diafiltration comprises about 20 volumes of said buffer exchange. In some embodiments, said buffer comprising a pH of about 9.0 to about 9.5 comprises a Tris concentration of about 40 mM to about 100 mM. In some embodiments, said second round of diafiltration comprises a buffer exchange with a buffer comprising a pH of about 7.0 to about 8.0. In some embodiments, said second round of diafiltration comprises about 10 volumes of said buffer exchange. In some embodiments, said buffer comprising a pH of about 7.0 to about 8.0 comprises a Tris concentration of about 15 mM to about 30 mM. In some embodiments, said buffer comprising a pH of about 7.0 to about 8.0 further comprises at least about 5% to at least about 20% sucrose, by volume. In some embodiments, said purified preparation of said recombinant oncolytic virus comprises from about 5 ng to about 100 ng of host cell DNA in a unit dose of said recombinant oncolytic virus. In some embodiments, said purified preparation of said recombinant oncolytic virus comprises from about 0.1 μg to about 10 μg of host cell protein in a unit dose of said recombinant oncolytic virus. In some embodiments, said unit dose comprises about 1×10⁹ to about 1×10¹³ PFU of said recombinant oncolytic virus. In some embodiments, said purified preparation of said recombinant oncolytic virus comprises up to about 400 ng, about 200 ng, about 100 ng, about 50 ng, or about 40 ng of host cell DNA per 5×10⁹ PFU of said recombinant oncolytic virus. In some embodiments, said purified preparation of said recombinant oncolytic virus comprises up to about 2000 ng, about 1000 ng, about 500 ng, about 250 ng, or about 200 ng of host cell DNA per 1×10⁹ PFU of said recombinant oncolytic virus. In some embodiments, said purified preparation of said recombinant oncolytic virus comprises up to about 7.5 μg, about 7 μg, about 6 μg, about 5 μg, or about 4 μg of host cell protein per 5×10⁹ PFU of said recombinant oncolytic virus. In some embodiments, said purified preparation of said recombinant oncolytic virus comprises up to about 37.5 μg, about 35 μg, about 30 μg, about 25 μg, or about 20 μg of host cell protein per 1×10⁹ PFU of said recombinant oncolytic virus. In some embodiments, said purified preparation of said recombinant oncolytic virus comprises up to about 100 ng, about 60 ng, about 50 ng, about 30 ng, or about 25 ng of serum albumin that is used in said culture per 5×10⁹ PFU of said recombinant oncolytic virus.

In another aspect, the present disclosure provides a method for producing a recombinant oncolytic virus, the method comprising: (i) contacting a culture of host cells in a bioreactor with a vaccinia virus at a multiplicity of infection of about 0.001 PFU/cell; (ii) growing said culture for a period of about 72 hours, at a temperature of about 36° C., and a dissolved oxygen tension level of about 50%, to generate a further culture comprising a plurality of virus infected host cells; (iii) harvesting from said further culture comprising said plurality of virus infected host cells a population of a recombinant oncolytic virus, and wherein said harvesting is carried out in presence of a buffer comprising a pH of about 9.5.

In another aspect, the present disclosure provides a method for producing a recombinant oncolytic virus, the method comprising: (i) contacting a culture of host cells in a bioreactor with a vaccinia virus at a multiplicity of infection of about 0.003 PFU/cell; (ii) growing said culture for a period of about 48 hours, at a temperature of about 35° C., and a dissolved oxygen tension level of about 45%, to generate a further culture comprising a plurality of virus infected host cells; (iii) harvesting from said further culture comprising said plurality of virus infected host cells a population of a recombinant oncolytic virus, wherein said bioreactor comprises a surface area of about 0.2 m2 to about 500 m2, and wherein said harvesting is carried out in presence of a buffer comprising a pH of about 8.5.

In another aspect, the present disclosure provides a method for producing a recombinant oncolytic virus, the method comprising: (i) contacting a culture of host cells in a bioreactor with a vaccinia virus at a multiplicity of infection of about 0.002 PFU/cell; (ii) growing said culture for a period of about 24 hours, at a temperature of about 32° C., and a dissolved oxygen tension level of about 50%, to generate a further culture comprising a plurality of virus infected host cells; (iii) harvesting from said further culture comprising said plurality of virus infected host cells a population of a recombinant oncolytic virus, wherein said bioreactor comprises a surface area of about 0.2 m2 to about 500 m2, and wherein said harvesting is carried out in presence of a buffer comprising a pH of about 8.0.

In another aspect, the present disclosure provides a method for producing a recombinant oncolytic virus, the method comprising: (i) contacting a culture of host cells in a bioreactor with a vaccinia virus at a multiplicity of infection of about 0.002 PFU/cell; (ii) growing said culture for a period of about 96 hours, at a temperature of about 37° C., and a dissolved oxygen tension level of about 50%, to generate a further culture comprising a plurality of virus infected host cells; (iii) harvesting from said further culture comprising said plurality of virus infected host cells a population of a recombinant oncolytic virus, wherein said bioreactor comprises a surface area of about 0.2 m2 to about 500 m2, and wherein said harvesting is carried out in presence of a buffer comprising a pH of about 7.8.

In another aspect, the present disclosure provides a method for producing a recombinant oncolytic virus, the method comprising: (i) contacting a culture of host cells in a bioreactor with a vaccinia virus at a multiplicity of infection of about 0.005 PFU/cell; (ii) growing said culture for a period of about 96 hours, at a temperature of about 37° C., and a dissolved oxygen tension level of about 50%, to generate a further culture comprising a plurality of virus infected host cells; (iii) harvesting from said further culture comprising said plurality of virus infected host cells a population of a recombinant oncolytic virus, wherein said bioreactor comprises a surface area of about 0.2 m2 to about 500 m2, and wherein said harvesting is carried out in presence of a buffer comprising a pH of about 9.5.

In another aspect, the present disclosure provides a method for producing a recombinant oncolytic virus, the method comprising: (i) contacting a culture of host cells in a bioreactor with a vaccinia virus at a multiplicity of infection of about 0.001 PFU/cell; (ii) growing said culture for a period of about 72 hours, at a temperature of about 36° C., and a dissolved oxygen tension level of about 50%, to generate a further culture comprising a plurality of virus infected host cells; (iii) harvesting from said further culture comprising said plurality of virus infected host cells a population of a recombinant oncolytic virus comprising at least about 1012 PFU of said recombinant oncolytic virus, and wherein said harvesting is carried out in presence of a buffer comprising a pH of about 9.5.

In another aspect, the present disclosure provides a method for producing a recombinant oncolytic virus, the method comprising: (i) contacting a culture of host cells in a bioreactor with a vaccinia virus at a multiplicity of infection of about 0.003 PFU/cell; (ii) growing said culture for a period of about 48 hours, at a temperature of about 35° C., and a dissolved oxygen tension level of about 45%, to generate a further culture comprising a plurality of virus infected host cells; (iii) harvesting from said further culture comprising said plurality of virus infected host cells a population of a recombinant oncolytic virus comprising at least about 1012 PFU of said recombinant oncolytic virus, wherein said bioreactor comprises a surface area of about 0.2 m2 to about 500 m2, and wherein said harvesting is carried out in presence of a buffer comprising a pH of about 8.5.

In another aspect, the present disclosure provides a method for producing a recombinant oncolytic virus, the method comprising: (i) contacting a culture of host cells in a bioreactor with a vaccinia virus at a multiplicity of infection of about 0.002 PFU/cell; (ii) growing said culture for a period of about 24 hours, at a temperature of about 32° C., and a dissolved oxygen tension level of about 50%, to generate a further culture comprising a plurality of virus infected host cells; (iii) harvesting from said further culture comprising said plurality of virus infected host cells a population of a recombinant oncolytic virus comprising at least about 1012 PFU of said recombinant oncolytic virus, wherein said bioreactor comprises a surface area of about 0.2 m2 to about 500 m2, and wherein said harvesting is carried out in presence of a buffer comprising a pH of about 8.0.

In another aspect, the present disclosure provides a method for producing a recombinant oncolytic virus, the method comprising: (i) contacting a culture of host cells in a bioreactor with a vaccinia virus at a multiplicity of infection of about 0.002 PFU/cell; (ii) growing said culture for a period of about 96 hours, at a temperature of about 37° C., and a dissolved oxygen tension level of about 50%, to generate a further culture comprising a plurality of virus infected host cells; (iii) harvesting from said further culture comprising said plurality of virus infected host cells a population of a recombinant oncolytic virus comprising at least about 1012 PFU of said recombinant oncolytic virus, wherein said bioreactor comprises a surface area of about 0.2 m2 to about 500 m2, and wherein said harvesting is carried out in presence of a buffer comprising a pH of about 7.8.

In another aspect, the present disclosure provides a method for producing a recombinant oncolytic virus, the method comprising: (i) contacting a culture of host cells in a bioreactor with a vaccinia virus at a multiplicity of infection of about 0.005 PFU/cell; (ii) growing said culture for a period of about 96 hours, at a temperature of about 37° C., and a dissolved oxygen tension level of about 50%, to generate a further culture comprising a plurality of virus infected host cells; (iii) harvesting from said further culture comprising said plurality of virus infected host cells a population of a recombinant oncolytic virus comprising at least about 1012 PFU of said recombinant oncolytic virus, wherein said bioreactor comprises a surface area of about 0.2 m2 to about 500 m2, and wherein said harvesting is carried out in presence of a buffer comprising a pH of about 9.5.

In some embodiments, the method further comprises purifying said population of recombinant oncolytic virus harvested in step (iii) to produce a purified preparation of said recombinant oncolytic virus. In some embodiments, said purified preparation of said recombinant oncolytic virus comprises at least about 2×10¹¹ to about 7×10¹¹ PFU of virus.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a flow chart of an exemplary virus manufacturing process.

FIG. 2 is a flow chart of an exemplary virus manufacturing process.

FIG. 3 is a flow chart of an exemplary virus manufacturing process.

FIG. 4 is a flow chart of an exemplary virus manufacturing process.

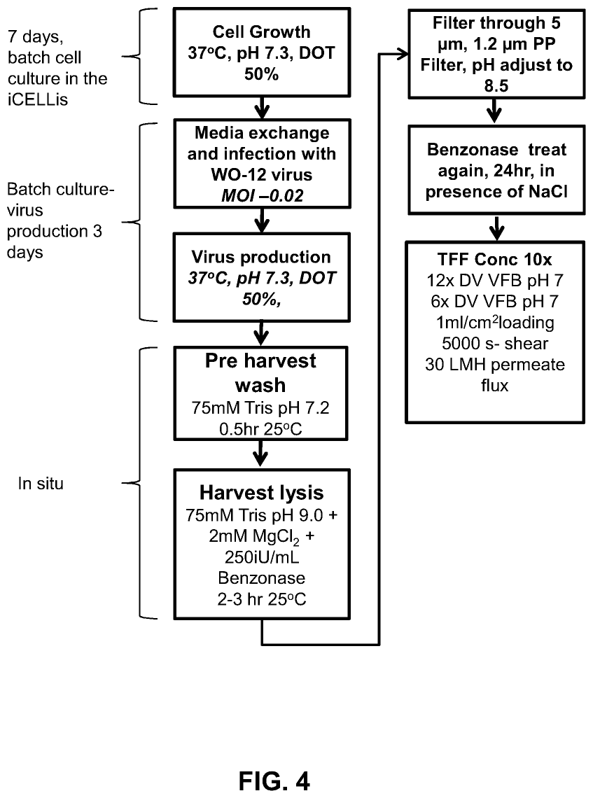

FIG. 5 is a flow chart of an exemplary virus manufacturing process.

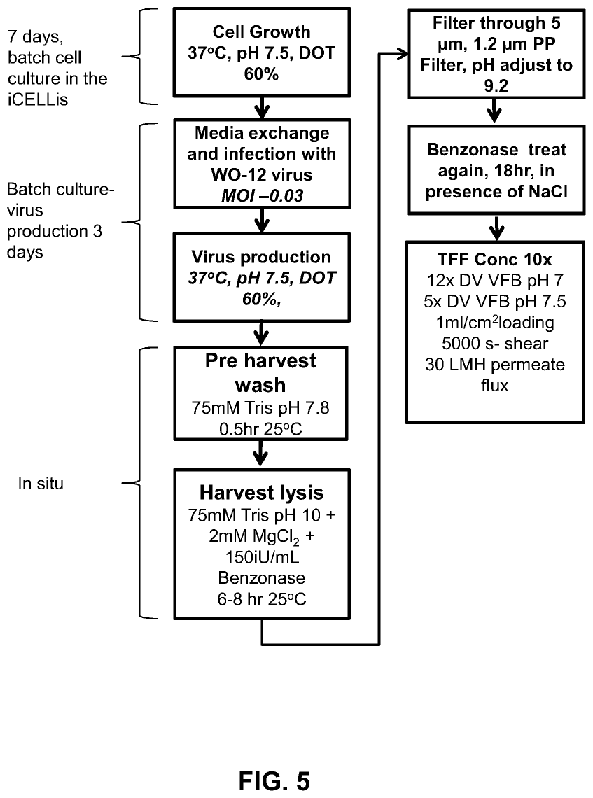

FIG. 6 is a flow chart of an exemplary downstream process for virus manufacturing.

FIG. 7 is a flow chart of an exemplary downstream process for virus manufacturing.

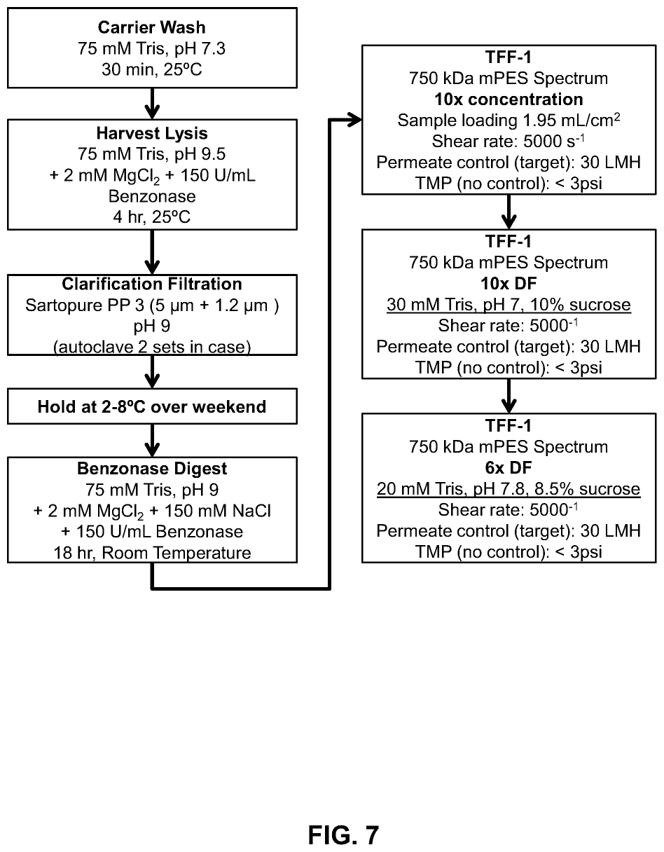

FIG. 8 is a flow chart of an exemplary downstream process for virus manufacturing.

FIG. 9 is a flow chart of an exemplary downstream process for virus manufacturing.

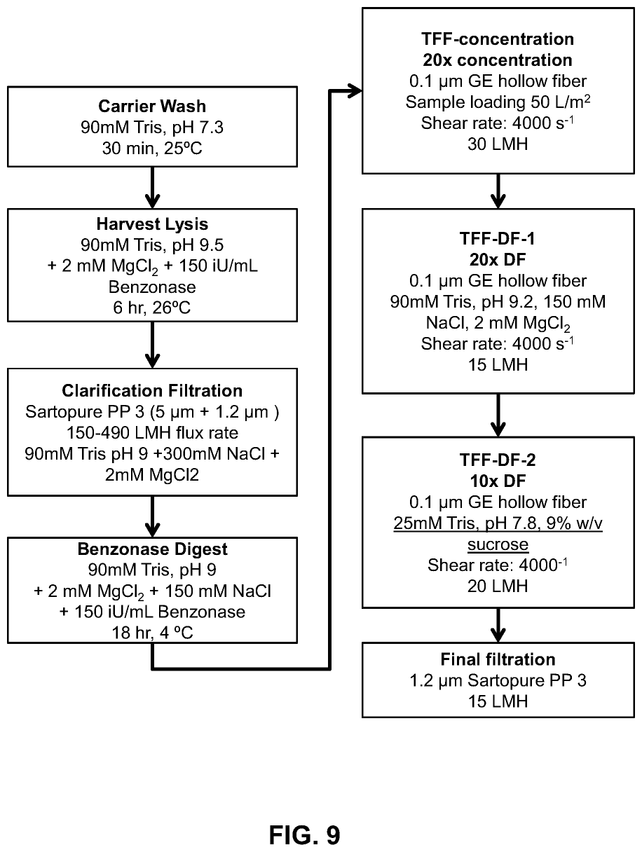

FIG. 10 shows a schematic of an exemplary setup for clarification process.

FIG. 11 shows a schematic of an exemplary setup for TFF and diafiltration process.

FIG. 12 shows a schematic of an exemplary setup for final filtration process.

### DETAILED DESCRIPTION

Certain Definitions

The terminology used herein is for the purpose of describing particular cases only and is not intended to be limiting. As used herein, the singular forms “a”, “an” and “the” can include the plural forms as well, unless the context clearly indicates otherwise. Furthermore, to the extent that the terms “contains,” “containing,” “including”, “includes,” “having,” “has”, “with”, or variants thereof are used in either the detailed description and/or the claims, such terms are intended to be inclusive in a manner similar to the term “comprising.”

The term “about” or “approximately” can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value, such as ±10% of the value modified by the term “about”.

The terms “individual”, “patient”, or “subject” can be used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker). In some embodiments, patients, subjects, or individuals can be under the supervision of a health care worker.

The term “mutation”, as used herein, can refer to a deletion, an insertion of a heterologous nucleic acid, an inversion or a substitution, including an open reading frame ablating mutations as commonly understood in the art.

The term “gene”, as used herein, can refer to a segment of nucleic acid that encodes an individual protein or RNA (also referred to as a “coding sequence” or “coding region”), optionally together with associated regulatory regions such as promoters, operators, terminators and the like, which may be located upstream or downstream of the coding sequence.

The terms “recombinant virus” and “modified virus”, as used interchangeably herein, can refer to a virus comprising one or more mutations in its genome, including but not limited to deletions, insertions of heterologous nucleic acids, inversions, substitutions or combinations thereof.

The term “oncolytic”, as used herein, can refer to killing of cancer or tumor cells by an agent, such as an oncolytic vaccinia virus, e.g., through the direct lysis of said cells, by stimulating immune response towards said cells, apoptosis, expression of toxic proteins, autophagy and shut-down of protein synthesis, induction of anti-tumoral immunity, or any combinations thereof. The direct lysis of the cancer or tumor cells infected by the agent, such as an oncolytic vaccinia virus, can be a result of replication of the virus within said cells. In certain examples, the term “oncolytic,” refers to killing of cancer or tumor cells without lysis of said cells.

The term “subject” can refer to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The terms “treat”, “treating”, and “treatment” can be meant to include alleviating or abrogating a disorder, disease, or condition; or one or more of the symptoms associated with the disorder, disease, or condition; or alleviating or eradicating the cause(s) of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

One aspect of the present disclosure provides methods and systems for manufacturing viruses. In some cases, the methods and systems provided herein can be efficient and productive in manufacturing viruses. For example, the methods and systems as described herein can produce viruses at a high titer per host cell. The methods and systems as described herein can be applied to produce viruses at an industrial scale, e.g., with a high yield per production cycle, e.g., from infection of host cell, culture of virus-infected cells, to harvest of viruses and clarification and purification of the viruses. The method provided herein can produce virus preparation of high purity, with limited amount to none of protein or nucleic acid contamination. In some cases, the methods and systems provided herein can be applied to produce recombinant viruses. In some cases, the methods and systems provided herein can be applied to produce oncolytic viruses.

A method of manufacturing virus as provided herein can comprise growing a cell culture comprising virus-infected host cells in a bioreactor. In some cases, methods and systems as provided herein can make use of fixed-bed bioreactor to culture virus-infected host cells. A method of manufacturing virus can comprise infecting (inoculating) host cells to generate a cell culture comprising virus-infected host cells. A method of manufacturing virus can comprise harvesting a population of viruses, e.g., oncolytic viruses, e.g., recombinant oncolytic viruses, from the cell culture. In some cases, harvesting the population of viruses can be performed in an alkaline condition. A method can comprise conducting clarification and purification of cell lysate obtained from the harvesting to generate a purified virus preparation. In some cases, the clarification and purification can comprise filtration of the cell lysate. In some cases, the clarification and purification can comprise treating the cell lysate with an enzyme to degrade cell debris followed by filtration. A method can also comprise conducting filtration to generate a concentrated virus preparation of high purity. In some cases, the filtration can comprise tangential flow filtration.

A method of manufacturing virus as provided herein can comprise (a) infecting (inoculating) a plurality of host cells to generate a cell culture comprising virus-infected host cells; (b) growing the cell culture comprising virus-infected host cells in a bioreactor; (c) harvesting a population of viruses, e.g., oncolytic viruses, e.g., recombinant oncolytic viruses, from the cell culture in an alkaline condition; and (d) conducting clarification and purification of cell lysate obtained from the harvesting to generate a purified virus preparation. In some cases, the clarification and purification can comprise: (i) treating the cell lysate with an enzyme to degrade cell debris; and (ii) conducting filtration of the treated cell lysate to generate a concentrated virus preparation of high purity.

A method as provided herein can comprise growing a cell culture to produce viruses, e.g., oncolytic viruses, e.g., recombinant oncolytic viruses, at a titer of at least about 50 PFU per host cell (PFU/cell), at least about 60 PFU/cell, at least about 70 PFU/cell, at least about 80 PFU/cell, at least about 90 PFU/cell, at least about 100 PFU/cell, at least about 110 PFU/cell, at least about 120 PFU/cell, at least about 140 PFU/cell, at least about 160 PFU/cell, at least about 180 PFU/cell, at least about 200 PFU/cell, at least about 220 PFU/cell, at least about 240 PFU/cell, at least about 250 PFU/cell, at least about 260 PFU/cell, at least about 280 PFU/cell, at least about 290 PFU/cell, at least about 300 PFU/cell, at least about 320 PFU/cell, at least about 340 PFU/cell, at least about 350 PFU/cell, at least about 360 PFU/cell, at least about 380 PFU/cell, at least about 400 PFU/cell, at least about 450 PFU/cell, at least about 500 PFU/cell, at least about 600 PFU/cell, at least about 700 PFU/cell, at least about 800 PFU/cell, at least about 900 PFU/cell, at least about 1000 PFU/cell, at least about 2000 PFU/cell, or at least about 5000 PFU/cell. In some cases, the method can comprise growing a cell culture to produce viruses, e.g., oncolytic viruses, e.g., recombinant oncolytic viruses, at a titer of about 50 PFU/cell to about 350 PFU/cell. In some cases, the method can comprise growing a cell culture to produce viruses at titer of about 100 PFU/cell to about 300 PFU/cell, 150 PFU/cell to about 300 PFU/cell, 200 PFU/cell to about 300 PFU/cell, 250 PFU/cell to about 300 PFU/cell, 100 PFU/cell to about 500 PFU/cell, 200 PFU/cell to about 500 PFU/cell, 250 PFU/cell to about 500 PFU/cell, 300 PFU/cell to about 500 PFU/cell, 400 PFU/cell to about 500 PFU/cell, 100 PFU/cell to about 1000 PFU/cell, 200 PFU/cell to about 1000 PFU/cell, 300 PFU/cell to about 1000 PFU/cell, 400 PFU/cell to about 1000 PFU/cell, 500 PFU/cell to about 1000 PFU/cell, 600 PFU/cell to about 1000 PFU/cell, 700 PFU/cell to about 1000 PFU/cell, 800 PFU/cell to about 1000 PFU/cell, or 900 PFU/cell to about 1000 PFU/cell.

A method as provided herein can comprise harvesting a population of viruses, e.g., oncolytic viruses, e.g., recombinant oncolytic viruses, from a culture that comprises about 1.5×10¹¹ to about 5×10¹³ PFU of the virus. In some cases, a method can comprise harvesting viruses from a culture that comprises at least about 1×10¹¹ PFU, at least about 1.5×10¹¹ PFU, at least about 2×10¹¹ PFU, at least about 2.5×10¹¹ PFU, at least about 3×10¹¹ PFU, at least about 4×10¹¹ PFU, at least about 5×10¹¹ PFU, at least about 7.5×10¹¹ PFU, at least about 1×10¹² PFU, at least about 1.5×10¹² PFU, at least about 2×10¹² PFU, at least about 2.5×10¹² PFU, at least about 3×10¹² PFU, at least about 4×10¹² PFU, at least about 5×10¹² PFU, at least about 7.5×10¹² PFU, at least about 1×10¹³ PFU, at least about 1.5×10¹³ PFU, at least about 2×10¹³ PFU, at least about 2.5×10¹³ PFU, at least about 3×10¹³ PFU, at least about 4×10¹³ PFU, at least about 5×10¹³ PFU, at least about 7.5×10¹³ PFU, or at least about 1×10¹⁴ PFU of the viruses. In some cases, a method can comprise harvesting viruses from a culture that can comprise about 1×10¹¹ to about 1×10¹⁴ PFU, about 5×10¹¹ to about 1×10¹⁴ PFU, about 1×10¹² to about 1×10¹⁴ PFU, about 5×10¹² to about 1×10¹⁴ PFU, about 1×10¹³ to about 1×10¹⁴ PFU, about 5×10¹³ to about 1×10¹⁴ PFU, about 1×10¹¹ to about 5×10¹³ PFU, about 2×10¹¹ to about 5×10¹³ PFU, about 5×10¹¹ to about 5×10¹³ PFU, about 1×10¹² to about 5×10¹³ PFU, about 2×10¹² to about 5×10¹³ PFU, about 5×10¹² to about 5×10¹³ PFU, or about 1×10¹³ to about 5×10¹³ PFU of the viruses.

A method as provided herein can comprise harvesting a population of viruses, e.g., oncolytic viruses, e.g., recombinant oncolytic viruses, from a culture that comprises the viruses at a viral titer of about 1.5×10⁸ to about 2×10¹⁰ PFU per mL of said culture, such as about 1.5×10⁸ to about 5×10⁸ PFU per mL, 2.5×10⁸ to about 7.5×10⁸ PFU per mL, 5×10⁸ to about 2.5×10⁹ PFU per mL, 1.5×10⁹ to about 5×10⁹ PFU per mL, 2.5×10⁹ to about 1×10¹⁰ PFU per mL, 5×10⁹ to about 1.5×10¹⁰ PFU per mL, or about 7.5×10⁹ to about 2×10¹⁰ PFU per mL of said culture.

A method as provided herein can comprise harvesting from a culture a population of viruses, e.g., oncolytic viruses, e.g., recombinant oncolytic viruses, to produce a lysate comprising the population of viruses, and clarifying the lysate to produce a population of purified viruses, the population of purified viruses can comprise at least about 50% to about 90% of the population of viruses in the lysate. In some cases, the population of purified viruses can comprise at least about 40%, at least about 50%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% of the population of viruses in the cell culture, e.g., oncolytic viruses, e.g., recombinant oncolytic viruses. In some cases, a, the population of purified viruses can comprise about 40% to about 99%, about 50% to about 99%, about 60% to about 99%, about 70% to about 99%, about 80% to about 99%, about 90% to about 99%, about 95% to about 99%, about 40% to about 90%, about 50% to about 90%, about 60% to about 90%, about 70% to about 90%, about 80% to about 90%, about 85% to about 90%, about 40% to about 80%, about 50% to about 80%, about 60% to about 80%, about 70% to about 80%, about 75% to about 80%, about 40% to about 50%, about 40% to about 60%, about 40% to about 70%, about 50% to about 60%, or about 50% to about 70% of the population of viruses in the cell culture, e.g., oncolytic viruses, e.g., recombinant oncolytic viruses.

A method as provided herein can comprise purifying the harvested virus lysate to generate a purified preparation of the virus. A purified preparation of the virus generated according to the methods provided herein can comprise at most about 2 ng, at most about 3 ng, at most about 4 ng, at most about 5 ng, at most about 6 ng, at most about 7 ng, at most about 8 ng, at most about 9 ng, at most about 10 ng, at most about 11 ng, at most about 12 ng, at most about 13 ng, at most about 14 ng, at most about 15 ng, at most about 16 ng, at most about 17 ng, at most about 18 ng, at most about 19 ng, at most about 20 ng, at most about 21 ng, at most about 22 ng, at most about 23 ng, at most about 24 ng, at most about 25 ng, at most about 28 ng, at most about 30 ng, at most about 50 ng, at most about 75 ng, at most about 100 ng, at most about 500 ng, at most about 750 ng, at most about 1 μg, or at most about 2 μg of host cell DNA in a unit dose of the virus. In some cases, a purified preparation of the virus generated according to the methods provided herein can comprise about 2 ng, about 3 ng, about 4 ng, about 5 ng, about 6 ng, about 7 ng, about 8 ng, about 9 ng, about 10 ng, about 11 ng, about 12 ng, about 13 ng, about 14 ng, about 15 ng, about 16 ng, about 17 ng, about 18 ng, about 19 ng, about 20 ng, about 21 ng, about 22 ng, about 23 ng, about 24 ng, about 25 ng, about 28 ng, about 30 ng, about 50 ng, about 82 mg, about 100 ng, about 200 ng, about 500 ng, about 750 ng, about 1 μg, or about 2 μg of host cell DNA in a unit dose of the virus. In some cases, the host cell DNA in the purified preparation of the virus can be measured by extraction and purification of DNAs using a silica-based column, or extraction of DNAs by standard methods. In some cases, the host cell DNA in the purified preparation of the virus can be measured after processing with QuickExtract™ or Qiagen® kits by quantitative real-time PCR with probes specific to the host cell genome. As provided herein, a unit dose of the virus can comprise about 0.5×10⁹, about 1×10⁹, about 2×10⁹, about 5×10⁹, about 7.5×10⁹, about 1×10¹⁰, about 2×10¹⁰, about 5×10¹⁰, about 7.5×10¹⁰, about 1×10¹¹, about 2×10¹¹, about 5×10¹¹, about 7.5×10¹¹, about 1×10¹², about 2×10¹², about 5×10¹², about 7.5×10¹², about 1×10¹³, about 2×10¹³, about 5×10¹³, about 7.5×10¹³, about 1×10¹⁴, about 2×10¹⁴, about 5×10¹⁴, about 7.5×10¹⁴, about 1×10¹⁵, or about 1×10¹⁶ PFU of the virus. In some case, a unit dose of the virus can comprise about 1×10⁹ to about 1×10¹³ PFU of the virus, e.g., oncolytic virus, e.g., recombination oncolytic virus. In some cases, a unit dose of the virus can comprise about 1×10⁹ to about 1×10¹³ viruses, such as 5×10⁹ viruses. In some examples, the unit dose of the virus can include intracellular mature virus (IMV), intracellular enveloped virus (IEV), cell-associated enveloped virus (CEV) and extracellular enveloped virus (EEV).

A purified preparation of the virus generated according to the methods provided herein can comprise at most about 5 ng, at most about 10 ng, at most about 15 ng, at most about 20 ng, at most about 25 ng, at most about 28 ng, at most about 30 ng, at most about 33 ng, at most about 35 ng, at most about 40 ng, at most about 45 ng, at most about 50 ng, at most about 80 ng, at most about 100 ng, at most about 150 ng, at most about 200 ng, at most about 250 ng, at most about 300 ng, at most about 400 ng, or at most about 500 ng of host cell DNA in dose of 5×10⁹ of the virus. In some cases, a purified preparation of the virus generated according to the methods provided herein can comprise about 5 ng, about 10 ng, about 15 ng, about 20 ng, about 25 ng, about 28 ng, about 30 ng, about 33 ng, about 35 ng, about 40 ng, about 45 ng, about 50 ng, about 80 ng, about 100 ng, about 150 ng, about 200 ng, about 250 ng, about 300 ng, about 400 ng, about 500 ng, about 1 μg, about 1.5 μg, about 2 μg, about 3 μg, or about 5 μg of host cell DNA in dose of 5×10⁹ of the virus. A purified preparation of the virus generated according to the methods provided herein can comprise at most about 1 ng, at most about 2 ng, at most about 3 ng, at most about 4 ng, at most about 5 ng, at most about 5.6 ng, at most about 6 ng, at most about 6.6 ng, at most about 7 ng, at most about 8 ng, at most about 9 ng, at most about 10 ng, at most about 16 ng, at most about 20 ng, at most about 30 ng, at most about 40 ng, at most about 50 ng, at most about 60 ng, at most about 80 ng, or at most about 100 ng, about 0.2 μg, about 0.3 μg, about 0.4 μg, or about 1 μg of host cell DNA in dose of 1×10⁹ of the virus. In some cases, a purified preparation of the virus generated according to the methods provided herein can comprise about 51 ng of host cell DNA in dose of 1×10⁹ of the virus.

A purified preparation of the virus generated according to the methods provided herein can comprise at most about 0.1 μg, at most about 0.2 μg, at most about 0.3 μg, at most about 0.4 μg, at most about 0.5 μg, at most about 0.6 μg, at most about 0.7 μg, at most about 0.8 μg, at most about 0.9 μg, at most about 1 μg, at most about 1.1 μg, at most about 1.2 μg, at most about 1.3 μg, at most about 1.4 μg, at most about 1.5 μg, at most about 2 μg, at most about 5 μg, at most about 7.5 μg, at most about 10 μg, at most about 20 μg, at most about 30 μg, at most about 50 μg, or at most about 100 μg of host cell protein in a unit dose of the virus. In some cases, a purified preparation of the virus generated according to the methods provided herein can comprise about 0.1 μg, about 0.2 μg, about 0.3 μg, about 0.4 μg, about 0.5 μg, about 0.6 μg, about 0.7 μg, about 0.8 μg, about 0.9 μg, about 1 μg, about 1.1 μg, about 1.2 μg, about 1.3 μg, about 1.4 μg, about 1.5 μg, about 2 μg, about 5 μg, about 7.5 μg, about 10 μg, about 20 μg, or about 30 μg of host cell protein in a unit dose of the virus. In some cases, a purified preparation of the virus generated according to the methods provided herein can comprise from about 0.1 μg to about 7.5 μg of host cell protein in a unit dose of said recombinant oncolytic virus. In some cases, the host cell protein content is determined by ELISA (enzyme-linked immunosorbent assay) targeting one or more host cell proteins.

In some cases, a purified preparation of the virus generated according to the methods provided herein can comprise at most about 0.1 μg, at most about 0.5 μg, at most about 1 μg, at most about 1.5 μg, at most about 2 μg, at most about 2.5 μg, at most about 3 μg, at most about 3.5 μg, at most about 4 μg, at most about 4.5 μg, at most about 5 μg, at most about 5.5 μg, at most about 6 μg, at most about 6.5 μg, at most about 7 μg, at most about 7.5 μg, at most about 10 μg, or at most about 20 μg of host cell protein in dose of 5×10⁹ of the virus. In some cases, a purified preparation of the virus generated according to the methods provided herein can comprise about 0.1 μg, about 0.5 μg, about 1 μg, about 1.5 μg, about 2 μg, about 2.5 μg, about 3 μg, about 3.5 μg, about 4 μg, about 4.5 μg, about 5 μg, about 5.5 μg, about 6 μg, about 6.5 μg, about 7 μg, about 7.5 μg, about 10 μg, or about 20 μg of host cell protein in dose of 5×10⁹ of the virus. In some cases, about 0.02 μg, at most about 0.1 μg, at most about 0.2 μg, at most about 0.3 μg, at most about 0.4 μg, at most about 0.5 μg, at most about 0.6 μg, at most about 0.7 μg, at most about 0.8 μg, at most about 0.9 μg, at most about 1 μg, at most about 1.1 μg, at most about 1.2 μg, at most about 1.3 μg, at most about 1.4 μg, at most about 1.5 μg, at most about 2 μg, or at most about 4 μg of host cell protein in dose of 1×10⁹ of the virus. In some cases, a purified preparation of the virus generated according to the methods provided herein can comprise about 0.02 μg, about 0.1 μg, about 0.2 μg, about 0.3 μg, about 0.4 μg, about 0.5 μg, about 0.6 μg, about 0.7 μg, about 0.8 μg, about 0.9 μg, about 1 μg, about 1.1 μg, about 1.2 μg, about 1.3 μg, about 1.4 μg, about 1.5 μg, about 2 μg, or about 4 μg of host cell protein in dose of 5×10⁹ of the virus.

Viruses

Methods and systems provided herein can be applicable to manufacturing a variety of different viruses, e.g., oncolytic viruses, e.g., recombinant oncolytic viruses. In some cases, the methods and systems provided herein can be applicable to manufacturing vaccinia viruses, e.g., recombinant vaccinia viruses.

Exemplary oncolytic viruses that the methods and system provided herein are applicable to include measles virus, poliovirus, poxvirus, vaccinia virus, an adenovirus, an adeno associated virus, herpes simplex virus, vesicular stomatitis virus, reovirus, Newcastle disease virus, Seneca virus, lentivirus, mengovirus, and myxoma virus. In certain examples, the oncolytic virus can be a vaccinia virus. In some cases, the methods and systems provided herein can be applicable to different strain variants of vaccinia viruses, such as, Lister, Dryvax, EM63, ACAM2000, Modified Vaccinia Ankara, LC16m8, CV-1, Western Reserve, Copenhagen, Connaught Laboratories, Wyeth, NYCBH, WRAE3L, and Dairen I. Viruses that the methods provided herein are applicable to can comprise genetic modifications, such as, deletion or mutation of one or more viral endogenous genes, or introduction of an exogenous virus. In some examples, the virus can be a recombinant vaccinia virus. In some cases, the recombinant vaccinia virus can comprise at least 2, 3, 4, 5, 6, 7, 8, 9, 10, 11, 12, 13, 14, 15, 20, or even more modifications in the genome of the virus. A deletion of a viral gene may include a partial or a complete deletion of the viral gene. It should be noted that as used herein, “partial deletion” or “mutation” can refer to an in situ partial deletion or mutation of an endogenous viral gene, respectively. Alternatively, they can refer to replacing the endogenous viral gene with an otherwise identical exogenous nucleic acid that lacks a portion of the gene (“partial deletion”) or has one or more nucleotide change in the gene (“mutation”).

In some cases, the virus that can be manufactured by the methods disclosed herein can be vaccinia virus comprising one or more genetic modifications. In some cases, the vaccinia virus can comprise TK (thymidine kinase) viral gene mutation, such that the vaccinia virus is thymidine kinase negative, C12L (IL18 binding protein) viral gene deletion, TRIF gene insertion, HPGD gene insertion, and reduced surface glycosylation. In some cases, the vaccinia virus can comprise at least one of the following engineered modifications: (i) C12L (IL18 binding protein) viral gene deletion; (ii) TRIF gene insertion; or (iii) HPGD gene insertion. In some cases, the vaccinia virus can comprise a reduced surface glycosylation. In some cases, the reduced surface glycosylation can be reduced sialylation of the surface of the virus. In some cases, the vaccinia virus can have a reduced glycosylation level as compared to a modified vaccinia virus. In some cases, the vaccinia virus can be treated with an agent that reduces the amount of glycosylation (e.g., sialylation) during or after the manufacture process as described herein but prior to administration to a host. In some cases, the vaccinia virus can further comprise one or more modifications selected from the group of a A34R Lys151 to Glu mutation; complete or partial deletion of B5R; mutation/deletion of A36R and/or mutation/deletion of A56R. In certain embodiments, the vaccinia virus can be a deglycosylated VV which comprises a complete or partial deletion of B5R.

Any suitable host cells can be chosen for manufacturing viruses according to the present disclosure. In some cases, the host cells used in the methods provided herein can be adherent cells. In other cases, the host cells can be cultured in suspension. In some cases, the host cells can be subcultured from an established cell line, such as, but not limited to, human embryonic kidney (HEK) cells such as HEK 293 cells, African green monkey kidney (AGMK) cells such as Vero cells, Chinese hamster ovary (CHO) cells, baby hamster kidney (BHK) cells, and Hela cells. In some cases, the host cells can be Hela cells. In some cases, the hots cells can be genetically modified cells, for instance, Hela cells that can be genetically modified by introducing one or more exogenous genes, or deletion or mutation of one or more endogenous genes. In some case, the host cells can be primary cells obtained from an organism, e.g., human. In some cases, the host cells can be immortalized cells that can be passaged (or subcultured) indefinitely.

In some examples, the method provided herein can comprise directly infecting the host cells with the virus, e.g., oncolytic virus, e.g., recombinant oncolytic virus. For example, the host cells can be brought into direct contact with the virus to become infected. In some cases, the viruses can be added into the culture medium of the host cells for inoculation. In some cases, the host cells can be infected by the virus at a multiplicity of infection (m.o.i.) of at least about 0.0005, at least about 0.00075, at least about 0.001, at least about 0.0015, at least about 0.002, at least about 0.003, at least about 0.005, at least about 0.0075, at least about 0.01, at least about 0.015, at least about 0.02, at least about 0.025, at least about 0.03, at least about 0.035, at least about 0.04, at least about 0.045, at least about 0.05, at least about 0.075, at least about 0.1, at least about 0.15, at least about 0.2, at least about 0.25, at least about 0.3, at least about 0.35, at least about 0.4, at least about 0.45, or at least about 0.5 PFU/cell. In some cases, multiplicity of infection can be calculated as plaque forming units of the virus used for infection divided by the number of host cells. In some cases, the host cells can be infected by the virus at a m.o.i. of at most about 0.0005, at most about 0.00075, at most about 0.001, at most about 0.0015, at most about 0.002, at most about 0.003, at most about 0.005, at most about 0.0075, at most about 0.01, at most about 0.015, at most about 0.02, at most about 0.025, at most about 0.03, at most about 0.035, at most about 0.04, at most about 0.045, at most about 0.05, at most about 0.075, at most about 0.1, at most about 0.15, at most about 0.2, at most about 0.25, at most about 0.3, at most about 0.35, at most about 0.4, at most about 0.45, or at most about 0.5 PFU/cell. In some cases, the host cells can be infected by the virus at a m.o.i. of about 0.0005, about 0.00075, about 0.001, about 0.0015, about 0.002, about 0.003, about 0.005, about 0.0075, about 0.01, about 0.015, about 0.02, about 0.025, about 0.03, about 0.035, about 0.04, about 0.045, about 0.05, about 0.075, about 0.1, about 0.15, about 0.2, about 0.25, about 0.3, about 0.35, about 0.4, about 0.45, or about 0.5 PFU/cell. In some cases, the host cells can be infected by the virus at an m.o.i. of about 0.001 to about 0.02 PFU/cell.

Culture of Virus-Infected Cells

Methods provided herein can comprise growing a culture comprising a plurality of virus-infected host cells in a bioreactor. Methods provided herein can produce viruses at high titer as described above. The bioreactor as used in the methods provided herein can be an integrated mixing system, offering even distribution of the culture medium. In some cases, the bioreactor can be capable of culturing cells at high density, leading to high production yield. In some cases, the bioreactor can have linear scalability, e.g., the configuration of the bioreactor can be adapted to both bench scale and industrial scale production depending on the need.

In some cases, the bioreactor can comprise a fixed-bed reactor. Methods and systems as provided herein can comprise a fixed bed reactor that comprise a vessel filled or packed with carrier materials that can be used as support for the immobilization of cells. The fixed bed reactor can also comprise a conditioning vessel that contains the culture medium. In some cases, the vessel with carrier materials and the conditioning vessel can be coupled via a circulation system. The circulation system can be a circulation loop. The circulation system can contain oxygen enriched medium that can be pumped from the conditioning vessel through the fixed-bed and back. In some cases, oxygen can be supplied directly into the fixed-bed. In a fixed bed reactor, the cells can be retained in the fixed-bed. The medium in the conditioning vessel can be exchanged either batch wise or continuously, so that the exhausted product-containing medium can be replaced with fresh medium. In some cases, the medium can be exchanged continuously. The fixed bed reactor can be an axial flow-based fixed bed system, in which an axial pumping of the media through the fixed bed can be used to provide the cells with fresh media and oxygen. In some cases, the fixed bed reactor can be a radial flow-based fixed bed system, in which a radial pumping of the media through the fixed bed, e.g., the media can be pumped through the fixed bed from a center point radially toward the surrounding the fixed bed. The fixed bed can comprise microcarriers. The microcarriers can be made of appropriate materials. In some cases, the microcarrier can be made of PET (polyethylene terephthalate). In some cases, the microcarrier can be made of synthetic materials, natural materials, or both. Synthetic materials include, but not limited to, glass (e.g., natron, borosilicate), DEAE-dextran, polypropylene, polyurethane, ceramic, acrylamide, polyhydroxyethylmethacrylate, polystyrene, and polyacrylamide. Natural materials include, but not limited to, collagen, cellulose, gelatin, fibrin, chitin and its derivatives, chitosan, alginate, polysaccharide, and polyglycan.

In some cases, the cell culture comprising virus-infected host cells can be grown in a fixed-bed bioreactor. The fixed-bed bioreactor can comprise microcarriers that can provide relatively large surface-to-volume ration with sufficient oxygen and nutrition exposure to the cells. The fixed-bed bioreactor can have a low draining time, e.g., time needed to drain up culture medium in the bioreactor to a maximum extent. In some cases, the draining time can be less than about 3 hours, less than about 2 hours, less than about 1 hours, less than about 30 min, less than about 20 min, less than about 15 min, less than about 14 min, less than about 13 min, less than about 12 min, less than about 11 min, or less than about 10 min. In some cases, the draining time can be about 80 min, about 50 min, about 30 min, about 12.5 min, about 12 min, about 11.5 min, about 11 min, or about 10.5 min. The fixed-bed bioreactor can have a low residual volume, e.g., the residual volume of culture medium when the medium can be drained to the maximum extent. In some cases, the bioreactor can have a residual volume of less than about 1500 ml, less than about 1200 ml, less than about 1000 ml, less than about 900 ml, less than about 800 ml, less than about 700 ml, less than about 600 ml, less than about 500 ml, less than about 400 ml, less than about 300 ml, less than about 200 ml, less than about 150 ml, or less than about 100 ml. In some cases, the bioreactor can have a residual volume of about 1150 ml, about 1100 ml, about 1050 ml, about 1000 ml, about 950 ml, about 900 ml, about 850 ml, about 800 ml, about 750 ml, about 700 ml, about 650 ml, about 600 ml, about 550 ml, about 500 ml, about 450 ml, about 400 ml, about 350 ml, about 300 ml, about 250 ml, about 200 ml, or about 150 ml.

In some cases, the method can comprise growing the culture in a fixed bed that comprises a volume of about 0.01 L to about 25 L. In some cases, the fixed bed can comprise a volume of at least about 0.005 L, at least about 0.01 L, at least about 0.05 L, at least about 0.1 L, at least about 0.5 L, at least about 1 L, at least about 2 L, at least about 3 L, at least about 4 L, at least about 5 L, at least about 6 L, at least about 7 L, at least about 8 L, at least about 9 L, at least about 10 L, at least about 12 L, at least about 14 L, at least about 15 L, at least about 16 L, at least about 18 L, at least about 20 L, at least about 21 L, at least about 22 L, at least about 23 L, at least about 24 L, at least about 25 L, at least about 26 L, at least about 27 L, at least about 28 L, at least about 29 L, at least about 30 L, at least about 35 L, at least about 40 L, at least about 50 L, at least about 75 L, or at least about 100 L. In some cases, the fixed bed can comprise a volume of about 0.005 L, about 0.01 L, about 0.05 L, about 0.1 L, about 0.5 L, about 1 L, about 2 L, about 3 L, about 4 L, about 5 L, about 6 L, about 7 L, about 8 L, about 9 L, about 10 L, about 12 L, about 14 L, about 15 L, about 16 L, about 18 L, about 20 L, about 21 L, about 22 L, about 23 L, about 24 L, about 25 L, about 26 L, about 27 L, about 28 L, about 29 L, about 30 L, about 35 L, about 40 L, about 50 L, about 60 L, about 65 L, about 70 L, about 75 L, or about 100 L. In some cases, the fixed bed can comprise a volume of about 1 L. In some cases, the fixed bed can comprise a volume of about 70 L. In some cases, the fixed bed can comprise a volume of about 0.005 L to about 25 L, about 0.01 L to about 25 L, about 0.05 L to about 25 L, about 0.1 L to about 25 L, about 0.15 L to about 25 L, about 0.2 L to about 25 L, about 0.5 L to about 25 L, about 1 L to about 25 L, about 5 L to about 25 L, about 10 L to about 25 L, about 15 L to about 25 L, about 20 L to about 25 L, about 0.005 L to about 15 L, about 0.05 L to about 15 L, about 0.5 L to about 15 L, about 5 L to about 15 L, about 10 L to about 15 L, about 0.005 L to about 50 L, about 0.5 L to about 50 L, about 5 L to about 50 L, about 10 L to about 50 L, about 15 L to about 50 L, about 25 L to about 50 L, about 30 L to about 50 L, about 1 L to about 100 L, about 5 L to about 100 L, about 10 L to about 100 L, about 20 L to about 100 L, about 50 L to about 100 L, or about 75 L to about 100 L.

In some cases, the method can comprise growing the culture in a fixed bed that comprises a compaction density of at least about 80 g/L to about 144 g/L. In some cases, the fixed bed can comprise a compaction density of at least about 20 g/L, at least about 30 g/L, at least about 40 g/L, at least about 50 g/L, at least about 60 g/L, at least about 70 g/L, at least about 80 g/L, at least about 90 g/L, at least about 91 g/L, at least about 92 g/L, at least about 93 g/L, at least about 94 g/L, at least about 95 g/L, at least about 96 g/L, at least about 97 g/L, at least about 98 g/L, at least about 99 g/L, at least about 100 g/L, at least about 110 g/L, at least about 120 g/L, at least about 130 g/L, at least about 140 g/L, at least about 150 g/L, or at least about 160 g/L. In some cases, the fixed bed can comprise a compaction density of at least about 96 g/L. In some cases, the fixed bed can comprise a compaction density of about 60 g/L, about 70 g/L, about 80 g/L, about 90 g/L, about 100 g/L, about 110 g/L, about 120 g/L, about 130 g/L, about 140 g/L, about 141 g/L, about 142 g/L, about 143 g/L, about 144 g/L, about 145 g/L, about 146 g/L, about 147 g/L, about 148 g/L, about 149 g/L, about 150 g/L, about 155 g/L, or about 160 g/L.

In some cases, the method can comprise seeding host cells in the bioreactor for growing for a certain period before virus inoculation. In other cases, the method can comprise seeding the virus infected host cells in the bioreactor for growing the culture. In some cases, the host cells prior to virus inoculation can be seeded at a density of at least about 1,000 cells/cm² to about 50,000 cells/cm² in the bioreactor. In some cases, the host cells prior to virus inoculation can be seeded at a density of at least about 10,000 cells/cm² to about 400,000 cells/cm². In some cases, the host cells prior to virus inoculation can be seeded at a density of at least about 1,000 cells/cm², at least about 1,500 cells/cm², at least about 2,000 cells/cm², at least about 2,500 cells/cm², at least about 3,000 cells/cm², at least about 3,500 cells/cm², at least about 4,000 cells/cm², at least about 4,500 cells/cm², at least about 5,000 cells/cm², at least about 10,000 cells/cm², at least about 20,000 cells/cm², at least about 40,000 cells/cm², at least about 50,000 cells/cm², at least about 60,000 cells/cm², at least about 70,000 cells/cm², at least about 80,000 cells/cm², at least about 90,000 cells/cm², at least about 100,000 cells/cm², at least about 110,000 cells/cm², at least about 120,000 cells/cm², at least about 130,000 cells/cm², at least about 140,000 cells/cm², at least about 150,000 cells/cm², at least about 160,000 cells/cm², at least about 170,000 cells/cm², at least about 180,000 cells/cm², at least about 190,000 cells/cm², at least about 200,000 cells/cm², at least about 210,000 cells/cm², at least about 220,000 cells/cm², at least about 240,000 cells/cm², at least about 260,000 cells/cm², at least about 280,000 cells/cm², at least about 300,000 cells/cm², at least about 340,000 cells/cm², at least about 360,000 cells/cm², at least about 380,000 cells/cm², at least about 400,000 cells/cm², at least about 450,000 cells/cm², or at least about 500,000 cells/cm². In some cases, the host cells can be seeded at a density of about 2,500 cells/cm². In some cases, the host cells prior to virus inoculation can be seeded at a density of about 1,500 cells/cm², about 1,600 cells/cm², about 1,800 cells/cm², about 2,200 cells/cm², about 2,200 cells/cm², about 2,400 cells/cm², about 2,600 cells/cm², about 2,800 cells/cm², about 3,000 cells/cm², about 3,500 cells/cm², about 4,000 cells/cm², about 4,500 cells/cm², about 5,000 cells/cm², about 6,000 cells/cm², about 7,000 cells/cm², about 8,000 cells/cm², about 9,000 cells/cm², about 10,000 cells/cm², about 15,000 cells/cm², about 20,000 cells/cm², about 30,000 cells/cm², about 40,000 cells/cm², about 50,000 cells/cm², about 60,000 cells/cm², about 70,000 cells/cm², about 80,000 cells/cm², about 90,000 cells/cm², about 100,000 cells/cm², about 110,000 cells/cm², about 120,000 cells/cm², about 130,000 cells/cm², about 140,000 cells/cm², about 150,000 cells/cm², about 160,000 cells/cm², about 170,000 cells/cm², about 180,000 cells/cm², about 190,000 cells/cm², about 200,000 cells/cm², about 210,000 cells/cm², about 220,000 cells/cm², about 240,000 cells/cm², about 260,000 cells/cm², about 280,000 cells/cm², about 300,000 cells/cm², about 340,000 cells/cm², about 360,000 cells/cm², about 380,000 cells/cm², about 400,000 cells/cm², about 450,000 cells/cm², or about 500,000 cells/cm².

In some cases, the method as described herein can comprise growing the host cells in the bioreactor for a determined period and then infecting the host cells at a determined density in the bioreactor. In some cases, the host cells can be grown in the bioreactor for about 5 days to about 15 days, such as about 5 days to about 8 days, about 7 days to about 10 days, about 8 days to about 12 days, or about 10 days to about 15 days. In some cases, the host cells can be grown in the bioreactor for about 5, 6, 7, 8, 9, or 10 days.

In some aspects, the present disclosure provides a bioreactor comprising a culture of host cells comprising a large number of viruses. In some cases, the bioreactor can be a bioreactor that can be used to culture virus-infected host cells according to the methods provided herein. An exemplary bioreactor can comprise a culture of a host cells comprising at least about 50 PFU to about 350 PFU of a recombinant oncolytic virus per host cell. Another exemplary bioreactor can comprise a culture of a host cells comprising at least about 1.5×10¹¹ to about 5×10¹³ PFU of a recombinant oncolytic virus. In some cases, the bioreactor can comprise a fixed bed, and the fixed bed can comprise the culture of host cells. In some cases, the culture of host cells can be propagated in microcarriers within the fixed bed. In some cases, the culture can comprise a host cell density of about 2,000 cells/cm² to about 10,000 cells/cm² of the fixed bed. In some cases, the fixed bed can comprise a volume of about 0.01 L to about 25 L. In some cases, the fixed bed can comprise a compaction density of at least about 80 g/L to about 144 g/L. In some cases, the fixed bed can comprise a surface area of about 0.2 m² to about 500 m², such as about 0.2 m² to about 0.5 m², about 0.4 m² to about 1 m², about 0.8 m² to about 1.5 m², about 1 m² to about 5 m², about 4 m² to about 6 m², about 5 m² to about 20 m², about 20 m² to about 50 m², about 50 m² to about 100 m², about 100 m² to about 200 m², about 200 m² to about 400 m², about 250 m² to about 500 m². In some cases, the fixed bed can comprise a surface area of about 4 m². In some cases, the fixed bed can comprise a surface area of about 500 m². Without wishing to be bound by a particular theory, the virus production scale can depend on the surface area of the fixed bed used in the method as described herein. For example, if a fixed bed having a surface area of about 4 m² is used to culture virus-infected cells, 4×10¹¹ to 1×10¹² PFU of virus can be produced using the method provided herein. In some cases, if a fixed bed having a surface area of about 500 m² is used to culture virus-infected cell, 1×10¹³ to 2.5×10¹⁴ PFU of virus can be produced using the method provided herein.

The method as provided herein can comprise growing a culture for a certain period of time, during which the viruses can replicate. The method as provided herein can comprise growing a culture for a sufficient period of time for reaching a desired viral titer in the culture. In some cases, the culture can be grown for about 24 hours to about 96 hours. In some cases, the culture can be grown for about 20 hours, about 22 hours, about 24 hours, about 26 hours, about 28 hours, about 30 hours, about 32 hours, about 34 hours, about 36 hours, about 38 hours, about 40 hours, about 42 hours, about 44 hours, about 46 hours, about 48 hours, about 50 hours, about 52 hours, about 54 hours, about 56 hours, about 58 hours, about 60 hours, about 62 hours, about 64 hours, about 66 hours, about 68 hours, about 70 hours, about 72 hours, about 74 hours, about 76 hours, about 78 hours, about 80 hours, about 82 hours, about 84 hours, about 86 hours, about 88 hours, about 90 hours, about 92 hours, about 94 hours, about 96 hours, about 100 hours, about 120 hours, about 150 hours, about 160 hours, or about 180 hours. In some cases, the culture can be grown for about 72 hours. In some cases, the culture can be grown for at least about 20 hours, at least about 24 hours, at least about 28 hours, at least about 30 hours, at least about 32 hours, at least about 36 hours, at least about 40 hours, at least about 42 hours, at least about 44 hours, at least about 46 hours, at least about 48 hours, at least about 52 hours, at least about 56 hours, at least about 60 hours, at least about 62 hours, at least about 64 hours, at least about 66 hours, at least about 68 hours, at least about 70 hours, at least about 72 hours, at least about 76 hours, at least about 80 hours, at least about 84 hours, at least about 88 hours, at least about 92 hours, at least about 94 hours, at least about 96 hours, at least about 100 hours, or at least about 120 hours. In some cases, the culture can be grown for at most about 24 hours, at most about 28 hours, at most about 32 hours, at most about 36 hours, at most about 40 hours, at most about 44 hours, at most about 48 hours, at most about 52 hours, at most about 56 hours, at most about 60 hours, at most about 64 hours, at most about 68 hours, at most about 72 hours, at most about 76 hours, at most about 80 hours, at most about 84 hours, at most about 88 hours, at most about 92 hours, at most about 96 hours, at most about 100 hours, at most about 120 hours, at most about 150 hours, at most about 160 hours, or at most about 180 hours. In some case, the culture can be grown for about 3 days.

The method as provided herein can comprise growing a culture at a dissolved oxygen tension (DOT) level of at least about 20% to about 100%. In some cases, the culture can comprise a DOT level of at least about 20%, at least about 30%, at least about 40%, at least about 45%, at least about 50%, at least about 60%, at least about 70%, at least about 80%, at least about 90%, or at least about 95%. In some cases, the culture can comprise a DOT level of about 20%, about 30%, about 40%, about 42%, about 44%, about 45%, about 46%, about 47%, about 48%, about 49%, about 50%, about 51%, about 52%, about 53%, about 54%, about 55%, about 56%, about 57%, about 58%, about 59%, about 60%, about 65%, about 70%, about 80%, about 90%, about 95%, or about 100%. In some cases, the culture can comprise a DOT level of about 50%. In some cases, the culture can comprise a DOT level of at most about 25%, at most about 30%, at most about 40%, at most about 50%, at most about 60%, at most about 70%, at most about 80%, at most about 90%, at most about 95%, or at most about 100%. In some case, the culture can comprise a DOT level of about 50%.

The method as provided herein can comprise growing a culture comprising the virus-infected host cells at a temperature of about 32° C. to about 38° C. The method as provided herein can comprise growing a culture comprising virus-infected host cells at a maintained temperature. In some cases, the maintained temperature can be about 32° C., about 33° C., about 34° C., about 35° C., about 36° C., about 36.2° C., about 36.4° C., about 36.6° C., about 36.8° C., about 37° C., about 37.2° C., about 37.4° C., about 37.6° C., about 37.8° C., or about 38° C. In some cases, the temperature of the culture can vary from time to time, e.g., at a first maintained temperature for a first period of time, and at a second maintained temperature for a second period of time, depending on the life cycle of the viruses, the growth stage of the host cells, or both. In some case, the maintained temperature can be controlled very precisely, for instance, with a variation less than ±1.5° C., less than ±1.2° C., less than ±1.0° C., less than ±0.9° C., less than ±0.8° C., less than ±0.7° C., less than ±0.6° C., less than ±0.5° C., less than ±0.4° C., less than ±0.3° C., less than ±0.2° C., less than ±0.1° C., or less than ±0.05° C. The precision at which the temperature of the culture can be maintained can be adjusted depending on a number of factors, such as, the life cycle of the viruses, the temperature sensitivity of the virus and the host cells, and the culture medium. In some cases, the culture can be maintained at about 37° C.

In some cases, the method provided herein can comprise growing a culture comprising virus-infected host cells at a pH of about 7.0 to about 7.5. In some cases, the pH of the culture can be maintained at a certain level, such as about 6.8, about 6.9, about 7.0, about 7.1, about 7.2, about 7.3, about 7.4, about 7.5, about 7.6, about 7.7, about 7.8, or about 8.0. In some cases, the maintained pH can be at least about 8.0, or at most about 7.0, depending on the type of the host cells and the viruses. In some cases, the pH of the culture can be maintained about 7.2. In some cases, the method can comprise growing a culture at a pH maintained with a variation of less than about 1.2, less than about 1.1, less than about 1.0, less than about 0.9, less than about 0.8, less than about 0.7, less than about 0.6, less than about 0.5, less than about 0.4, less than about 0.3, less than about 0.2, less than about 0.1, less than about 0.08, less than about 0.06, less than about 0.05, less than about 0.02, or less than about 0.01. During cell culture, the pH of the culture comprising virus-infected host cells can be adjusted by exchanging the medium with fresh medium that can be well pH-adjusted. The pH of the culture can be maintained by pH buffer. In some cases, the pH can be adjusted by CO₂ gas control or by supplementing acid or alkaline into the culture medium, for instance, adding HCl to reduce the pH, or adding NaOH to increase the pH, to a desired level. In some aspects, pH can also be modified by adding CO₂.

Harvest of Viruses

Some aspects of the present disclosure provide a method of harvesting viruses, e.g., oncolytic viruses, e.g., recombinant oncolytic viruses, from a culture comprising a plurality of virus-infected host cells. Harvesting viruses can comprise lysis of the virus-infected host cells, thereby releasing the viruses into the lysate, and collecting the lysate. Harvesting viruses can also comprise pre-harvest wash of the bioreactor. In some cases, the pre-harvest wash can be conducted in order to remove contaminants (e.g., proteins, nucleic acids, exhaust products, and other molecules in the culture medium) from the lysate.

After culturing the virus-infected host cells for a certain period of time as described above, the bioreactor, e.g., the carriers, can be washed by an appropriate wash solution, in preparation for the subsequent virus harvesting. In some cases, the wash solution can be a buffer solution, such as Tris buffer solution. In some cases, the wash solution can comprise 75 mM Tris and a pH of 7.3. The washing step can be performed at any appropriate temperature for any appropriate duration. In some cases, the parameters of the washing step, such as, the wash solution (e.g., ionic concentration, pH), temperature, washing duration, can be well adjusted so that the host cells can be rarely lysed at this step. In some cases, the carriers can be washed by 75 mM Tris solution (pH 7.3, 25° C.) for 30 min.

In some aspects, the present disclosure provides a pH shift method for harvesting viruses from the culture. Harvesting according to some aspects of the present disclosure can comprise incubating the virus-infected host cells in an alkaline buffer. In some cases, harvesting also can comprise incubating the virus-infected host cells with an enzyme to degrade cell debris. In some cases, harvesting can comprise incubating the virus-infected host cells with an enzyme to degrade cell debris and an alkaline buffer. The alkaline buffer can comprise a pH of at least about 8. In some cases, the alkaline buffer can comprise a pH of at least about 8.0, at least about 8.2, at least about 8.4, at least about 8.5, at least about 8.6, at least about 8.8, at least about 9.0, at least about 9.1, at least about 9.2, at least about 9.3, at least about 9.4, at least about 9.5, at least about 9.6, at least about 9.7, at least about 9.8, at least about 9.9, at least about 10.0, at least about 10.4, at least about 10.8, or at least about 11.0. In some cases, the alkaline buffer can comprise a pH of about 8.0, about 8.2, about 8.4, about 8.5, about 8.6, about 8.8, about 8.9, about 9.0, about 9.1, about 9.2, about 9.3, about 9.4, about 9.5, about 9.6, about 9.7, about 9.8, about 9.9, about 10.0, about 10.2, about 10.4, about 10.6, about 10.8, or about 11.0. In some cases, the alkaline buffer can comprise Tris. In some cases, the alkaline buffer can comprise a pH of about 9.5. In some cases, the alkaline buffer can comprise a pH of about 9.6. In some cases, the alkaline buffer can have a determined pH with a variation of less than about 0.2, less than about 0.1, less than about 0.08, less than about 0.06, less than about 0.05, less than about 0.02, or less than about 0.01. In one example, the alkaline buffer can comprise 75 mM Tris, 2 mM MgCl₂ and pH of 9.5. In another example, the alkaline buffer can comprise 90 mM Tris, 2 mM MgCl₂ and pH of 9.5. In one example, the alkaline buffer can comprise 75 mM Tris, 2 mM MgCl₂ and pH of 9.6. In one example, the alkaline buffer can comprise 75 mM Tris, 2.5 mM MgCl₂ and pH of 9.6. Without wishing to be bound to a particular theory, high pH of the lysis buffer can contribute to better lysis of the host cells, the endosomes of the host cells, or both, so that the viruses that can be “trapped” therein can be more easily exposed, thereby leading to a higher yield of viruses.

In some cases, an enzyme can be used to degrade cell debris in the method provided herein. The enzyme can comprise nuclease, e.g., endonuclease for clearing free nucleic acids in the cell lysate. In some cases, the enzyme can comprise proteases, e.g., TrypLE or trypsin.

A nuclease can refer to an enzyme capable of cleaving the phosphodiester bonds between monomers of nucleic acids. Nucleases can cause single and double stranded breaks in their target molecules. Different nucleases, such as exonuclease and endonuclease can be used in the methods provided herein, which act on different loci of a nucleic acid target. Exonucleases can digest nucleic acids from the ends, while endonucleases can act on regions in the middle of target molecules. The nucleases as used herein can be naturally-occurring nucleases, or recombinant (engineered) nucleases, whose amino acid sequences deviate from naturally-occurring nucleases. For example, genetically engineered Serratia nuclease (e.g., Benzonase®) can be used for this purpose. In some cases, the method can comprise incubating the virus-infected host cells with an enzyme to degrade cell debris that can be about 50 IU/mL to about 200 IU/mL. In some cases, during the incubation of virus harvesting, the enzyme to degrade cell debris can be about 20 IU/mL, about 30 IU/mL, about 50 IU/mL, about 75 IU/mL, about 100 IU/mL, about 125 IU/mL, about 130 IU/mL, about 140 IU/mL, about 150 IU/mL, about 160 IU/mL, about 170 IU/mL, about 180 IU/mL, about 190 IU/mL, about 200 IU/mL, about 220 IU/mL, about 240 IU/mL, about 260 IU/mL, about 280 IU/mL, about 300 IU/mL, or about 400 IU/mL. In some cases, during the incubation of virus harvesting, the enzyme to degrade cell debris can be at least about 20 IU/mL, at least about 30 IU/mL, at least about 50 IU/mL, at least about 75 IU/mL, at least about 100 IU/mL, at least about 125 IU/mL, at least about 130 IU/mL, at least about 140 IU/mL, at least about 150 IU/mL, at least about 160 IU/mL, at least about 170 IU/mL, at least about 180 IU/mL, at least about 190 IU/mL, at least about 200 IU/mL, at least about 220 IU/mL, or at least about 240 IU/mL. In some cases, during the incubation of virus harvesting, the enzyme to degrade cell debris can be at most about 50 IU/mL, at most about 75 IU/mL, at most about 100 IU/mL, at most about 125 IU/mL, at most about 130 IU/mL, at most about 140 IU/mL, at most about 150 IU/mL, at most about 160 IU/mL, at most about 170 IU/mL, at most about 180 IU/mL, at most about 190 IU/mL, at most about 200 IU/mL, at most about 220 IU/mL, at most about 240 IU/mL, at most about 260 IU/mL, at most about 280 IU/mL, at most about 300 IU/mL, or at most about 400 IU/mL. In some embodiments of the method provided herein, the virus harvesting can comprise incubating the virus-infected host cells with Benzonase® at about 150 IU/mL.

The incubation of the virus-infected host cells in a cell lysis buffer for virus harvesting can last for an appropriate amount of time. In some cases, the incubation can be for about 2 hours to about 8 hours. In some cases, the incubation can be for about 1 hour, about 2 hours, about 3 hours, about 4 hours, about 5 hours, about 6 hours, about 7 hours, about 8 hours, about 9 hours, about 10 hours, about 12 hours, about 15 hours, or about 20 hours. In some cases, the incubation can be for at least about 2 hours to at least about 8 hours. In some cases, the incubation can be for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 12 hours, or at least about 15 hours. In some cases, the incubation can be for about 2 hours to about 8 hours. In some cases, the incubation can be for at most about 2 hours, at most about 3 hours, at most about 4 hours, at most about 5 hours, at most about 6 hours, at most about 7 hours, at most about 8 hours, at most about 9 hours, at most about 10 hours, at most about 12 hours, at most about 15 hours, or at most about 20 hours. In some embodiments of the method provided herein, the virus harvesting can comprise incubating the virus-infected host cells in a cell lysis buffer for about 4 hours.

The incubation of the virus-infected host cells in a cell lysis buffer for virus harvesting can be at any appropriate temperature, for instance, about 22° C. to about 28° C. In some cases, the temperature for the incubation for virus harvesting can be at about 22° C., at about 23° C., at about 24° C., at about 25° C., at about 26° C., at about 27° C., at about 28° C., at about 29° C., at about 30° C., at about 31° C., or at about 32° C. In some cases, the temperature can be maintained at a certain degree. In some case, the maintained temperature can be controlled very precisely, for instance, with a variation less than ±1.5° C., less than ±1.2° C., less than ±1.0° C., less than ±0.9° C., less than ±0.8° C., less than ±0.7° C., less than ±0.6° C., less than ±0.5° C., less than ±0.4° C., less than ±0.3° C., less than ±0.2° C., less than ±0.1° C., or less than ±0.05° C. In some embodiments of the method provided herein, the virus harvesting can comprise incubating the virus-infected host cells at about 25° C.

Clarification

In some cases, the method provided herein can further comprise conducting a clarification step to generate a filtered lysate. The step of clarification can be performed to eliminate or reduce contaminants and purify the virus. In some cases, the step of clarification as provided herein can comprise filtration and nuclease treatment. In some cases, the step of clarification can comprise purification of the virus by centrifugation.

The step of clarification can comprise filtration. In some cases, the lysate harvested by the method provided herein can be subject to filtration through a filter to generate a filtered lysate. As provided herein, a filter can have a pore size that retains particles of a size larger than the pore size and passes particles of a size smaller than the pore size. Any appropriate filter can be chosen to conduct the filtration step for the purpose of isolating and purifying the virus. In some cases, the filter can have an average filter size that can be at least about 0.15 μm, at least about 0.18 μm, at least about 0.2 μm, at least about 0.22 μm, at least about 0.25 μm, at least about 0.28 μm, at least about 0.3 μm, at least about 0.35 μm, at least about 0.4 μm, at least about 0.45 μm, at least about 0.5 μm, at least about 0.55 μm, at least about 0.6 μm, at least about 0.65 μm, at least about 0.7 μm, at least about 0.75 μm, at least about 0.8 μm, at least about 0.85 μm, at least about 0.9 μm, at least about 0.95 μm, or at least about 1 μm. In some cases, the filter can have an average filter size that can be at most about 0.18 μm, at most about 0.2 μm, at most about 0.22 μm, at most about 0.25 μm, at most about 0.28 μm, at most about 0.3 μm, at most about 0.35 μm, at most about 0.4 μm, at most about 0.45 μm, at most about 0.5 μm, at most about 0.55 μm, at most about 0.6 μm, at most about 0.65 μm, at most about 0.7 μm, at most about 0.75 μm, at most about 0.8 μm, at most about 0.85 μm, at most about 0.9 μm, at most about 0.95 μm, at most about 1 μm, or at most about 1.5 μm. In some cases, filtration can be performed with more than one filter, e.g., 2, 3, 4, 5, 6, or more filters. In some cases, the more than one filter can have the same pore size or different sizes, for instance, in some examples, the lysate can be filtered by a filter with average size of about 5 μm followed by a filter with average size of about 1.2 μm. In some examples, the lysate can be filtered by a filter with average size of about 5 μm followed by a filter with average size of about 2.4 μm, and then about a filter about 1 μm.

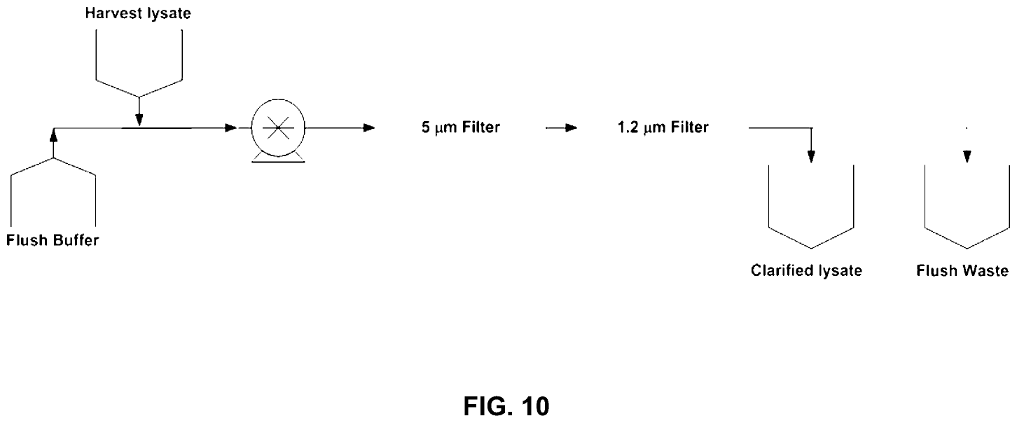

As exemplified in FIG. 10, the clarification process can be carried out by flushing the lysate through a filter with the aid of a flush buffer. In some cases, the flow rate for the clarification process can be about 50 to about 500 mL/min, such as about 50 to about 100 mL/min, about 75 to about 120 mL/min, about 100 to about 150 mL/min, about 125 to about 175 mL/min, about 150 to about 200 mL/min, about 175 to about 250 mL/min, about 225 to about 300 mL/min, about 250 to about 325 mL/min, about 275 to about 350 mL/min, about 300 to about 375 mL/min, about 325 to about 400 mL/min, about 350 to about 425 mL/min, about 375 to about 450 mL/min, about 400 to about 475 mL/min, or about 425 to about 500 mL/min. In some cases, the flow rate for the clarification process can be about 70, 80, 90, 100, 110, 120, 130, 150, 175, 200, 225, 250, 275, 280, 290, 300, or 320 mL/min. Flow rate can sometimes be described in LMH (L/m²/h). In some cases, the flow rate for the clarification process can be about 100 to about 750 LMH, such as about 100 to about 175 LMH, about 125 to about 200 LMH, about 150 to about 225 LMH, about 175 to about 250 LMH, about 200 to about 275 LMH, about 225 to about 300 LMH, about 250 to about 325 LMH, about 275 to about 350 LMH, about 300 to about 375 LMH, about 325 to about 400 LMH, about 350 to about 425 LMH, about 375 to about 450 LMH, about 400 to about 475 LMH, about 425 to about 500 LMH, about 450 to about 525 LMH, about 475 to about 550 LMH, about 500 to about 575 LMH, or about 525 to about 600 LMH. In some cases, the flow rate for the clarification process can be about 150 LMH. In some cases, the flow rate for the clarification process can be about 490 LMH. In some cases, the flush rate can be chosen differently for filters of different pore sizes, for example, a higher flow rate can be used for filter of a larger pore size whereas a lower flow rate can be used for filter of a smaller pore size. In some cases, the flush buffer can have similar constituents as the lysate, for example, similar ions and ionic concentrations, and similar pH, or different constituents from the lysate. In some cases, the clarification process can be carried out at room temperature, or any other appropriate temperature, for instance, at about 20° C., 21° C., 22° C., 23° C., 24° C., 25° C., 28° C., 30° C., 32° C., or 37° C.

After the filtration step, the filtered lysate can be further treated with an enzyme to degrade cell debris, e.g., a nuclease, e.g., Benzonase®, to eliminate contaminants, e.g., nucleic acids, and to generate a depth filtrate. In some cases, the filtered lysate can be incubated with a buffer containing Benzonase® for a sufficient amount of time. In some cases, the filtered lysate can be incubated with a buffer containing at least about 10 U/ml, at least about 20 U/ml, at least about 40 U/ml, at least about 50 U/ml, at least about 60 U/ml, at least about 80 U/ml, at least about 100 U/ml, at least about 120 U/ml, at least about 130 U/ml, at least about 140 U/ml, at least about 150 U/ml, at least about 160 U/ml, at least about 170 U/ml, at least about 180 U/ml, at least about 200 U/ml, at least about 250 U/ml, at least about 300 U/ml, at least about 500 U/ml, or at least about 1000 U/ml Benzonase®. In some cases, the treatment of the filtered lysate by the enzyme to degrade cell debris can be conducted at pH of at least about 8.0, at least about 8.1, at least about 8.2, at least about 8.3, at least about 8.4, at least about 8.5, at least about 8.6, at least about 8.7, at least about 8.8, at least about 8.9, at least about 9.0, at least about 9.1, at least about 9.2, at least about 9.3, at least about 9.4, at least about 9.5, at least about 9.6, at least about 9.7, at least about 9.8, or at least about 10.0. In some cases, the treatment of the filtered lysate by the enzyme to degrade cell debris can be conducted at pH of about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, or about 10.0. In some cases, the treatment of the filtered lysate by the enzyme to degrade cell debris can be conducted at pH of about 7.5 to about 9.5, about 7.5 to about 10.0, about 8.0 to about 10.0, about 8.5 to about 10.0, about 8.5 to about 10.5, about 9.0 to about 10.5, or about 9.0 to about 11.0. In some cases, the filtered lysate can be treated by the enzyme to degrade cell debris for at least about 1 hour, at least about 2 hours, at least about 4 hours, at least about 6 hours, at least about 8 hours, at least about 10 hours, at least about 12 hours, at least about 14 hours, at least about 16 hours, at least about 18 hours, at least about 20 hours, at least about 22 hours, at least about 24 hours, at least about 26 hours, at least about 28 hours, at least about 30 hours, at least about 34 hours, at least about 36 hours, or at least about 48 hours. In some cases, the filtered lysate can be treated by the enzyme to degrade cell debris for about 1 hour, about 2 hours, about 4 hours, about 6 hours, about 8 hours, about 10 hours, about 12 hours, about 14 hours, about 16 hours, about 18 hours, about 20 hours, about 22 hours, about 24 hours, about 26 hours, about 28 hours, about 30 hours, about 34 hours, about 36 hours, or about 48 hours. In some cases, the filtered lysate can be treated by the enzyme to degrade cell debris for at most about 3 hours, at most about 5 hours, at most about 7 hours, at most about 9 hours, at most about 11 hours, at most about 13 hours, at most about 15 hours, at most about 17 hours, at most about 19 hours, at most about 21 hours, at most about 23 hours, at most about 25 hours, at most about 27 hours, at most about 29 hours, at most about 31 hours, at most about 35 hours, at most about 40 hours, or at most about 50 hours. In some cases, the filtered lysate can be treated with Benzonase® for about 18 hours at room temperature. In some cases, the enzyme treatment can be conducted in the presence of a monocation, e.g., Na⁺, e.g., sodium chloride (NaCl) in the solution. In some cases, the enzyme treatment can be conducted in the presence of high concentration of monocation, e.g., Na⁺, e.g., sodium chloride (NaCl), in the solution, for instance, at least about 100 mM, at least about 120 mM, at least about 150 mM, at least about 180 mM, at least about 200 mM, at least about 250 mM, at least about 300 mM, at least about 350 mM, at least about 400 mM, at least about 450 mM, at least about 500 mM, at least about 550 mM, at least about 600 mM, at least about 700 mM, at least about 800 mM, at least about 900 mM, or at least about 1000 mM. In some cases, the enzyme treatment can be conducted in the presence of high concentration of monocation, e.g., Na⁺, e.g., sodium chloride (NaCl), in the solution, for instance, about 100 mM, about 120 mM, about 150 mM, about 180 mM, about 200 mM, about 250 mM, about 300 mM, about 350 mM, about 400 mM, about 450 mM, about 500 mM, about 550 mM, about 600 mM, about 700 mM, about 800 mM, about 900 mM, or about 1000 mM.

In some cases, according to some aspects of the methods provided herein, the virus lysate, either unfiltered or filtered, can be stored at a low temperature before the next step. In some cases, the filtered lysate can be saved at low temperature before the treatment by enzyme to degrade cell debris. For example, the filtered lysate can be stored at about 2 to 8° C. for a certain period of time, e.g., 6 hours, 12 hours, 24 hours, 2 days, 3 days, or 6 days.

Purification

In some cases, the methods provided herein can comprise conducting tangential flow filtration (TFF) of the filtered lysate to generate a purified preparation of said recombinant oncolytic virus. In some cases, tangential flow filtration can be followed by diafiltration to further purify the virus. In some cases, more than one round of TFF or diafiltration can be conducted. In some cases, 2 rounds of diafiltration can be conducted. In some cases, 2, 3, 4, 5, or more rounds of TFF or diafiltration can be conducted. In other cases, other techniques can be used in the subject methods for purifying the viruses as an alternative of or in combination with the TFF process as described herein, for instance, ultrafiltration or size-exclusion chromatography (for example, with Sephadex® G25 or Sephadex® G10 or equivalent materials), ion-exchange chromatography, affinity chromatography, size exclusion chromatography or “reversed-phase” chromatography.

Tangential Flow Filtration (TFF), also known as crossflow filtration, can refer to a filtration process where the feed stream passes parallel to the filter membrane face as one portion passes through the membrane (permeate) while the remainder (retentate) can be recirculated back to the feed reservoir. As provided herein, TFF can be performed to concentrate the filtered lysate. In some cases, after TFF, the filtered lysate can be concentrated by at least 2 times, at least 3 times, at least 4 times, at least 5 times, at least 6 times, at least 7 times, at least 8 times, at least 9 times, at least 10 times, at least 12 times, at least 15 times, at least 18 times, at least 20 time, or at least 30 times. In some cases, after TFF, the filtered lysate can be concentrated by about 2 times, about 3 times, about 4 times, about 5 times, about 6 times, about 7 times, about 8 times, about 9 times, about 10 times, about 12 times, about 15 times, about 18 times, about 20 time, or about 30 times. In some cases, after TFF, the filtered lysate can be concentrated by about 10 times.

In some cases, a TFF filter membrane having a pore size that can be about 300 kDa, about 350 kDa, about 400 kDa, about 450 kDa, about 500 kDa, about 550 kDa, about 600 kDa, about 650 kDa, about 700 kDa, about 750 kDa, about 800 kDa, about 850 kDa, about 900 kDa, or about 1000 kDa. In some cases, a TFF filter membrane having a pore size that can be at most about 300 kDa, at most about 350 kDa, at most about 400 kDa, at most about 450 kDa, at most about 500 kDa, at most about 550 kDa, at most about 600 kDa, at most about 650 kDa, at most about 700 kDa, at most about 750 kDa, at most about 800 kDa, at most about 850 kDa, at most about 900 kDa, or at most about 1000 kDa.

Appropriate TFF filter and filtration parameters can be selected and adjusted depending on a number of parameters, including, but not limited to, the type of virus to be manufactured, the type of host cells, cell culture medium, and filter buffer. In some cases, the TFF process can be carried out at any appropriate temperature, such as at about 20° C., 21° C., 22° C., 23° C., 24° C., 25° C., 28° C., 30° C., 32° C., or 37° C. In some cases, the loading for TFF can be at least about 10 L/m², at least about 20 L/m², at least about 30 L/m², at least about 40 L/m², at least about 50 L/m², at least about 60 L/m², at least about 70 L/m², at least about 80 L/m², at least about 100 L/m², at least about 120 L/m², at least about 150 L/m², or at least about 200 L/m². In some cases, the loading for TFF can be about 10 L/m², about 20 L/m², about 30 L/m², about 40 L/m², about 50 L/m², about 60 L/m², about 70 L/m², about 80 L/m², about 100 L/m², about 120 L/m², about 150 L/m², or about 200 L/m². In some cases, the TFF process can be carried out at shear rate of about 2000 to about 6000 s⁻¹, such as about 2000 to about 3500 s⁻¹, about 2500 to about 4000 s⁻¹, about 3000 to about 4500 s⁻¹, about 3000 to about 5000 s⁻¹, about 3500 to about 5000 s⁻¹, about 3500 to about 5500 s⁻¹, about 4000 to about 6000 s⁻¹, or about 4500 to about 6000 s⁻¹. In some cases, the TFF process can be carried out at shear rate of about 2000 s⁻¹, about 2500 s⁻¹, about 3000 s⁻¹, about 3500, about 3500 s⁻¹, about 4000 s⁻¹, about 4500 s⁻¹, about 5000 s⁻¹, about 5500 s⁻¹, about 6000 s⁻¹. In some cases, during the TFF process, the lysate can be flowed at a rate of about 0.25 to about 2.5 L/min, such as about 0.25 to about 0.75 L/min, about 0.5 to about 1 L/min, about 0.75 to about 1.25 L/min, about 1 to about 1.5 L/min, about 1.25 to about 1.75 L/min, about 1.5 to about 2.0 L/min, about 1.75 to about 2.25 L/min, or about 2 to about 2.5 L/min. In some cases, the flow rate and the shear rate can be chosen in combination, for instance, lysate can be flowed at about 1.5 L/min at a shear rate of 5000 s⁻¹, or about 0.8 L/min at a shear rate of 3000 s⁻¹. In some cases, permeate flux rate can be controlled throughout the TFF process. In some cases, the permeate flux rate can be controlled at between 5 and about 50 LMH, such as between 5 and about 15 LMH, between 10 and about 20 LMH, between 15 and about 25 LMH, between 20 and about 30 LMH, between 25 and about 35 LMH, between 30 and about 40 LMH, between 35 and about 45 LMH, or between 40 and about 50 LMH. In some cases, the permeate flux rate can be controlled at between about 15 to about 20 LMH. In some cases, the permeate flux rate can be controlled at between about 10 to about 30 LMH. In some cases, the permeate flux rate can be controlled at between about 15 to about 30 LMH. In some cases, the permeate flux rate can be controlled at between about 15 LMH. In some cases, the permeate flux rate can be controlled at between about 20 LMH.

In some cases, the method can further comprise diafiltration of the filtered lysate or the purified lysate. In some cases, diafiltration can be performed to remove salts or other contaminants from the lysate to generate a purified virus preparation. As provided herein, diafiltration can be a fractionation process that washes smaller molecules through a membrane and leaves larger molecules in the retentate without ultimately changing concentration.

Either continuous diafiltration or discontinuous diafiltration can be used in the method provided herein. In continuous diafiltration, the diafiltration buffer can be added to the feed reservoir at the same rate as a filtrate can be generated. In this way the volume in the sample reservoir can remain constant, but the small molecules (e.g. salts) that can freely permeate through the membrane can be washed away. As a result, each additional volume of buffer exchange can reduce the salt concentration further. As provided herein, a “volume of buffer exchange” can be equal to the volume of the starting solution before the diafiltration solution can be added. In discontinuous diafiltration, the starting solution can be first diluted and then concentrated back to the starting volume. This process can then be repeated until the desirable concentration of small molecules (e.g. salts) remaining in the reservoir can be reached. In some cases, the diafiltration process can be carried out using the same TFF system for the earlier concentration process. In some cases, the diafiltration process can be carried out using a different system. As provided herein, a diafiltration can comprise adding at least 2, at least 3, at least 4, at least 5, at least 6, at least 7, at least 8, at least 9, at least 10, at least 12, at least 14, at least 15, at least 16, at least 18, at least 20, at least 21, at least 22, at least 23, at least 25, or at least 30 volumes of buffer exchange. In some cases, a diafiltration as provided herein can comprise 6 volumes of buffer exchange. In some cases, a diafiltration as provided herein can comprise 10 volumes of buffer exchange. In some cases, a diafiltration as provided herein can comprise 20 volumes of buffer exchange. In some cases, the methods provided herein can comprise 2 rounds of diafiltration, a first round having 10 volumes of buffer exchange followed by a second round having 6 volumes of buffer exchange, or a first round having 20 volumes of buffer exchange followed by a second round having 10 volumes of buffer exchange. In some cases, the first round of buffer exchange can be carried with a buffer having similar constituents to the lysate, whereas the second round of buffer exchange can be carried out with a buffer used to filter out the ions contained in the lysate, for example a buffer containing sucrose. In some cases, the buffer exchange can be conducted with a buffer comprising a pH of about 7.8. In some cases, the buffer exchange can be conducted with a buffer comprising a pH of about 7.0. In some cases, the buffer exchange can be conducted with a buffer comprising a pH of about 7.0, about 7.1, about 7.2, about 7.4, about 7.5, about 7.6, about 7.7, about 7.8, about 7.9, about 8.0, about 9.0, or about 10.0. In some cases, the buffer exchange can be conducted with a buffer comprising a pH of at least about 7.0, at least about 7.1, at least about 7.2, at least about 7.4, at least about 7.5, at least about 7.6, at least about 7.7, at least about 7.8, at least about 7.9, at least about 8.0, at least about 9.0, or at least about 10.0. In some cases, the buffer exchange can be conducted with a buffer comprising a pH of about 7 or about 7.8. In some cases, the buffer exchange can be conducted with a Tris buffer. The Tris buffer can comprise about 5 mM, about 10 mM, about 12 mM, about 15 mM, about 20 mM, about 22 mM, about 24 mM, about 25 mM, about 26 mM, about 28 mM, about 30 mM, about 35 mM, or about 40 mM Tris. In some examples, the buffer exchange can be conducted with a buffer comprising about 30 mM, or about 20 mM Tris. In some examples, the buffer exchange can be conducted with a buffer. Water can be solvent of the buffer. And the buffer can comprise uncharged molecules, like saccharide, such as sucrose or glucose, histidine, sorbitol. The buffer can comprise, in some cases, ionic constituents, like MgCl₂ or NaCl, or other stabilizing molecules. In some cases, the buffer for diafiltration can comprise about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 13%, about 14%, about 15%, about 20%, or about 30% weight by volume of sucrose. In some cases, the buffer exchange can be conducted with a buffer comprising about 10% or about 8.5% weight by volume of sucrose. In some cases, additional rounds of buffer exchange can be performed to achieve a desired concentration. For example, a target concentration can be 0.5×, 1×, 1.25×, 1.5×, 1.75×, 2×, 2.25×, 2.5×, 2.75×, 3×, 4×, 5×, 6×, 7×, 8×, 9×, 10×, 11×, 12×, 13×, 14×, 15×, 16×, 17×, 18×, 19×, or up to about 20×.

In some cases, additional clarification or filtration steps can be performed depending on the need for purity, concentration, or titer of the virus product. In some cases, for example, a final filtration step can be performed in which TFF retentate can be further filtered through a filter with the aid of a final formulation buffer. The formulation buffer can comprise ion constituents as needed for the final virus product, for instance. Any appropriate flow parameters can be chosen as described above. A filter of relatively small pore size, for instance, 1.2 μm, can be used for the final step. Filters of a variety of pore sizes can be utilized in any aspect of the present disclosure. In some aspects, a filter can have a pore size of about 0.1 μm, 0.2 μm, 0.3 μm, 0.4 μm, 0.5 μm, 0.6 μm, 0.7 μm, 0.8 μm, 0.9 μm, 1.0 μm, 1.1 μm, 1.2 μm, 1.3 μm, 1.4 μm, 1.5 μm, 1.6 μm, 1.7 μm, 1.8 μm, 1.9 μm, 2.0 μm, 2.5 μm, 3.0 μm, 4.0 μm, or up to about 5.0 μm.

### EXAMPLES

The following examples are offered by way of illustration, not by way of limitation.

### Example 1. Upstream Process (I)

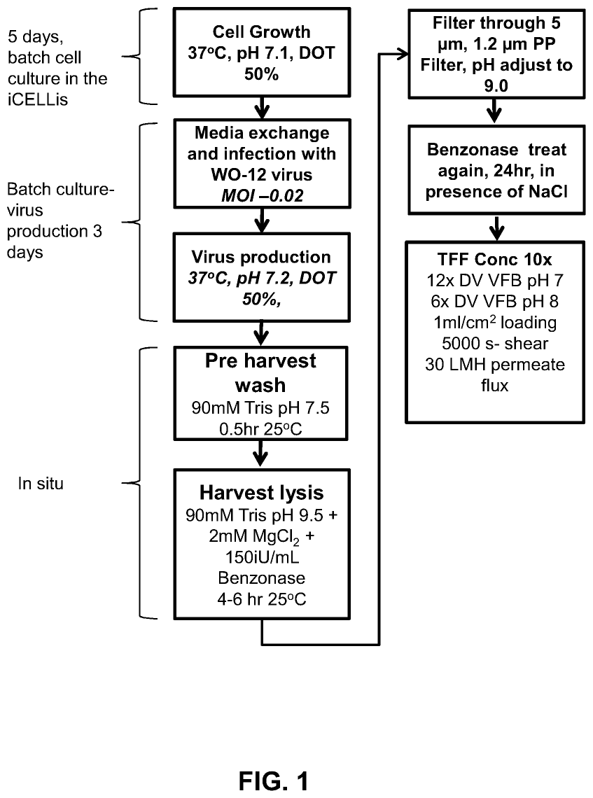

This example describes an exemplary protocol for upstream processes, as depicted in the flow chart in FIG. 1, for manufacturing a recombinant vaccinia virus from culture of virus-infected host cells, harvesting, to cell lysis.

1. Host Hela cells are cultured in iCELLis nano bioreactor (Pall Corporation, Port Washington, N.Y.) for 5 days at 37° C. pH of the culture medium is maintained at 7.1, and the dissolved oxygen tension (DOT) level of the culture medium is 50%;

2. On day 6, culture medium is exchange with culture medium containing initial recombinant vaccinia virus at multiplicity of infection (m.o.i.) of 0.02. After infection, the virus-infected Hela cells continued to be cultured at 37° C., pH of 7.2, and DOT of 50% for 3 days;

3. On day 9, the bioreactor is pre-washed before harvesting, the culture medium is exchanged with a wash buffer containing 90 mM Tris and having pH of 7.5 for 0.5 hr at 25° C.;

4. After pre-wash, the wash buffer is exchanged with lysis medium containing 90 mM Tris, 2 mM MgCl2, and 150 iU/mL Benzonase®, and having pH of 9.5. The virus-infected cells are incubated with the lysis medium for 4-6 hours at 25° C., after which the lysis medium containing the cell lysate is collected for further processing.

### Example 2. Upstream Process (II)

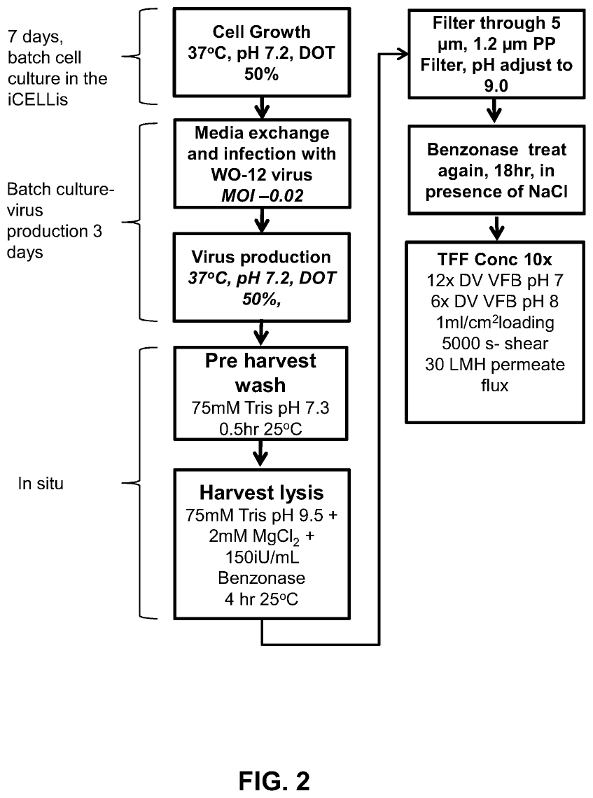

This example describes an exemplary protocol for upstream processes, as depicted in the flow chart in FIG. 2, for manufacturing a recombinant vaccinia virus from culture of virus-infected host cells, harvesting, to cell lysis.

1. Host Hela cells are cultured in iCELLis nano bioreactor (Pall Corporation, Port Washington, N.Y.) for 7 days at 37° C. pH of the culture medium is maintained at 7.2, and the dissolved oxygen tension (DOT) level of the culture medium is 50%;

2. On day 8, culture medium is exchange with culture medium containing initial recombinant vaccinia virus at multiplicity of infection (m.o.i.) of 0.02. After infection, the virus-infected Hela cells continued to be cultured at 37° C., pH of 7.2, and DOT of 50% for 3 days;

3. On day 11, the bioreactor is pre-washed before harvesting, the culture medium is exchanged with a wash buffer containing 75 mM Tris and having pH of 7.3 for 0.5 hr at 25° C.;

4. After pre-wash, the wash buffer is exchanged with lysis medium containing 75 mM Tris, 2 mM MgCl2, and 150 iU/mL Benzonase®, and having pH of 9.5. The virus-infected cells are incubated with the lysis medium for 4 hours at 25° C., after which the lysis medium containing the cell lysate is collected for further processing.

### Example 3. Upstream Process (III)

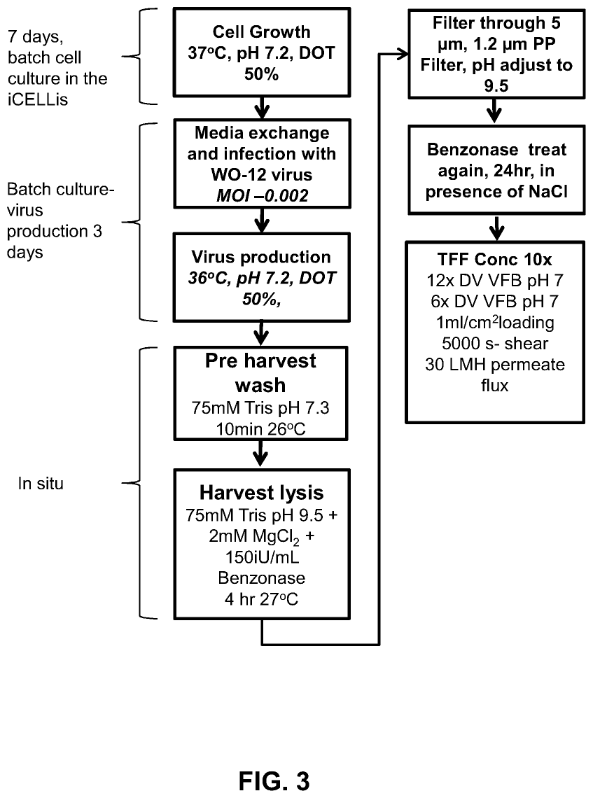

This example describes an exemplary protocol for upstream processes, as depicted in the flow chart in FIG. 3, for manufacturing a recombinant vaccinia virus from culture of virus-infected host cells, harvesting, to cell lysis.

1. Host Hela cells are cultured in iCELLis nano bioreactor (Pall Corporation, Port Washington, N.Y.) for 7 days at 37° C. pH of the culture medium is maintained at 7.2, and the dissolved oxygen tension (DOT) level of the culture medium is 50%;

2. On day 8, culture medium is exchange with culture medium containing initial recombinant vaccinia virus at multiplicity of infection (m.o.i.) of 0.002. After infection, the virus-infected Hela cells continued to be cultured at 36° C., pH of 7.2, and DOT of 50% for 3 days;

3. On day 11, the bioreactor is pre-washed before harvesting, the culture medium is exchanged with a wash buffer containing 75 mM Tris and having pH of 7.3 for 10 min at 26° C.;

4. After pre-wash, the wash buffer is exchanged with lysis medium containing 75 mM Tris, 2 mM MgCl₂, and 150 iU/mL Benzonase®, and having pH of 9.5. The virus-infected cells are incubated with the lysis medium for 4 hours at 25° C., after which the lysis medium containing the cell lysate is collected for further processing.

### Example 4. Downstream Process (I)

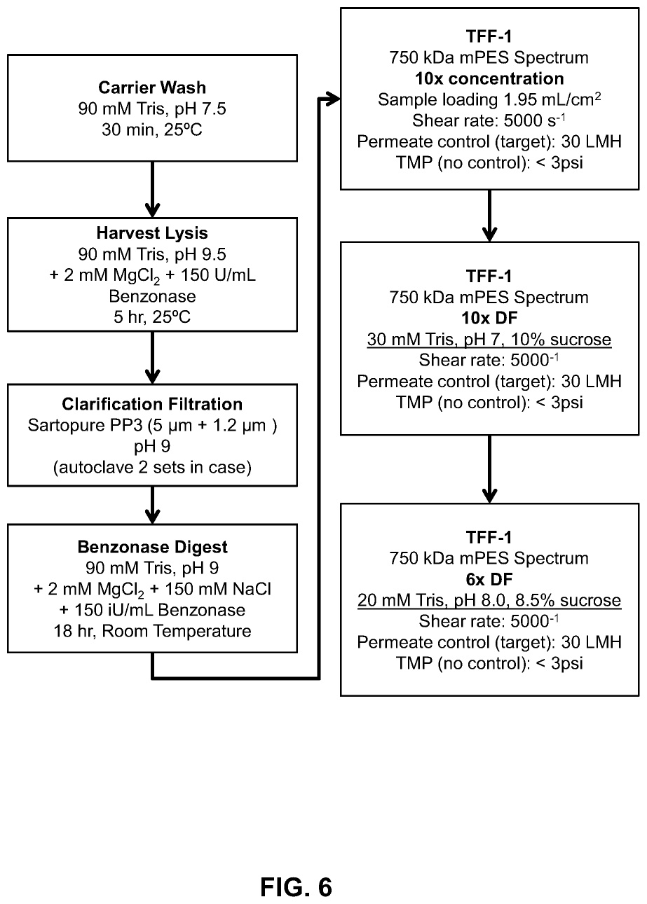

This example describes an exemplary protocol for downstream process, as depicted in FIG. 6, for producing an exemplary vaccinia virus.

Harvesting Virus after culturing the virus-infected host Hela cells in an iCELLis nano bioreactor for 3 days.

1. Control sample collection: as a control, sample ten carriers from the reactor bed and place the sample in 15 mM Tris, pH 8.0 (0.05 ml/cm²) lysis buffer for 30 min (volume of lysis buffer: 7 ml).

2. Drain culture medium for pre-wash, and turn off pumps and removed all medium according to procedure:

(a) Keep medium out loop running and run medium in counterclockwise direction until unprimed;

(b) Turn medium out off;

(c) Discard medium in the recirculation bottle (open and pour out);

(d) Stop regulation;

(e) Close all clamps;

(f) Open flow path from sample (2 clamps) bottle and open gas out clamp;

(g) Using giant syringe (or hand pump), on gas out line, pressure pump medium into sampling bottle.

3. Wash bed.

(a) Clamp off recirculation loop;

(b) Add through sample bottle 800 ml 25° C. wash buffer containing 90 mM Tris and having pH of 7.5±0.2, at a linear flow rate of 1 cm/sec and 25° C. for 0.5 hr;

(c) Discard wash buffer after incubation.

4. During the wash, collect a sample to measure pH offline. Perform pH adjustment if necessary.

5. Just before removing the wash buffer out of the bioreactor, collect another sample and read offline pH to confirm iCELLis online pH is reading correctly.

6. Set the pH control of the bioreactor to 9.5±0.2.

7. Set the temp control of the bioreactor to 25° C.

8. Add 600 ml lysis buffer containing 90 mM Tris and 150 U Benzonase®/ml and having pH 9.0±0.2, and add 2.4 ml of 0.5M MgCl2.

9. Wait until the pH drops and stabilizes.

10. Take an offline pH measure and adjust iCELLis offset if necessary:

Turn on pH control (set point 9.0±0.2), and increase linear flow rate to 1.25 cm/sec and let run for 4.0 hr at 25° C.

11. Lysis time starts when the bioreactor indicates that pH reaches 9.0. Take another offline pH reading to verify it's reading correctly.

12. 2 hr after lysis starts, take sample.

13. Drain the bioreactor 4 hr after lysis starts.

14. Measure the pH and adjust if necessary (use 1M HCL if pH is too high).

Record if adjusted.

Clarification

1. Set up a filtering system as depicted in FIG. 10.

2. Prime the Sartopure PP3 (5.0 um/1.2 um) filters by flushing the filters with 750 mL priming buffer (75 mM Tris, pH 9, 2 mM MgCl2) by using a peristaltic pump at 250 ml/min:

(a) Open the air ports on both filters and flush until buffer comes out and then close off until fully primed;

(b) Pump until all priming buffer is gone and open the air ports again (no more liquid should come out).

3. Filter the entire lysate by pumping the lysate through the filter using a peristaltic pump at 250 ml/min.

(c) Begin to slowly pump in the viral lysate until liquid comes out of the side air ports and then cap off and wipe with ethanol;

(d) Pump all of the lysate (˜600 ml) through the filters at a rate of 250 ml/min.

(e) At the end, pump through until only air comes out of the air ports (open);

(f) Separate this vessel and collect sample.

4. Flush the filter with an equal volume of flush buffer (75 mM Tris, pH 9, 2 mM MgCl2) at 250 ml/min into a separate container, and sample the flush buffer.

5. Pool the filtered lysate and flush buffer, collect another sample.

6. Immediately adjust pH of the pooled lysate of step 4 to 9 using a small amount of 1M HCl.

7. Store the pH9.0 pooled filtrate at 2-8° C. (over the weekend if needed): if store over the weekend, take 6×250 μl samples before preceding to the next step.

8. Further lysis to produce depth filtrate: add 150 iU/ml of Benzonase® (720 ul) plus enough 2M NaCl (90 mL) to reach 500 mM NaCl and pH 9.0, and incubate at room temperature overnight 18±4 hrs.

TFF and Diafiltration

1. Set up a sterile 750 kD mPES (modified Polyethersulfone), 790 cm2 hollow fiber filter (Spectrum) in KrosFlo® TFF system.

2. Pre-flush the TFF system with a pre-flush buffer (75 mM Tris pH 9, 2 mM MgCl2) until everything is primed. Flush out the 60-70 ml hold volume in the system, and discard the pre-flush buffer.

3. Place the depth filtrate on a balance and connect the tubing to the inlet and outlet lines of the TFF system.

4. Recirculate the sample for 10 min, and ensure the shear is approximately 4000-5000 sec-1.

5. Run the depth filtrate through the filter membrane (aim to stay around or below 5000 sec-1 shear rate, TMP (transmembrane pressure) ˜1.2 psid), and slowly open the permeate line until the crossflow is no more than 30 LMH (L/m2/h). Run until the sample has been concentrated by about 10 times. The final volume should be about 100-120 ml in the reservoir bottle.

7. Begin the first round (Virus Formulation Buffer “VFB”, pH 7) of diafiltration by opening the VFB line and maintaining constant weight in the sample vessel (aim for 5000 sec-1 shear rate, maximum 25-30 LMH, ˜960 ml/min), do 10 volumes of buffer exchange with VFB pH 7.

8. Begin the final round (20 mM Tris, pH 7.8, 8.5% sucrose) of diafiltration by opening the VFB line and maintaining constant weight in the sample vessel (again aim for 5000 sec-1 shear rate, maximum 25 LMH), and do 6 volumes of buffer exchange with 20 mM Tris, pH 8, 8.5% sucrose.

9. Perform the final concentration step until there is only 15-30 mL left in the reservoir, and then flush the system until the hold volume is added to the reservoir (60-70 ml, resulting in total of 75-100 ml at the end).

10. Store the final retentate in approximately 5 ml aliquots in 50 ml conical tubes at −80° C.

### Example 5. Downstream Process (II)

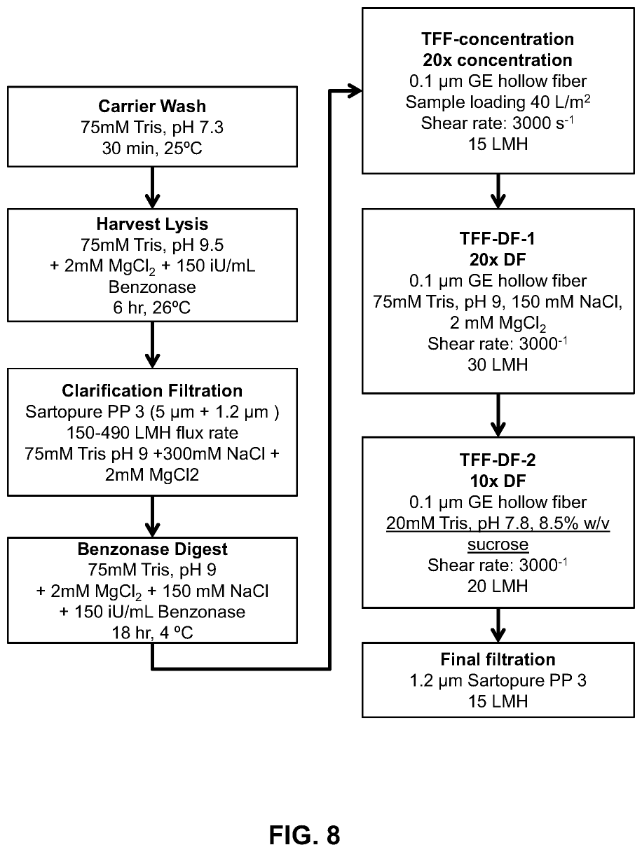

This example describes an exemplary protocol for downstream process, as depicted in FIG. 8, for producing an exemplary vaccinia virus. The records listed below are records taken from an experimental run of the protocol.

Harvesting Virus after culturing the virus-infected host Hela cells in an iCELLis nano bioreactor for 3 days.

1. Control sample collection: as a control, sample ten carriers from the reactor bed and place the sample in 15 mM Tris, pH 8.0 (0.05 ml/cm²) lysis buffer for 30 min (volume of lysis buffer: 7 ml).

2. Drain culture medium for pre-wash, and turn off pumps and removed all medium according to procedure:

(a) Keep medium out loop running and run medium in counterclockwise direction until unprimed;

(b) Turn medium out off;

(c) Discard medium in the recirculation bottle (open and pour out);

(d) Stop regulation;

(e) Close all clamps;

(f) Open flow path from sample (2 clamps) bottle and open gas out clamp;

(g) Using giant syringe (or hand pump), on gas out line, pressure pump medium into sampling bottle.

3. Wash bed.

(a) Clamp off recirculation loop;

(b) Add through sample bottle 800 ml 25° C. wash buffer containing 75 mM Tris and having pH of 7.3±0.2, at a linear flow rate of 1 cm/sec and 25° C. for 0.5 hr;

(c) Discard wash buffer after incubation.

4. During the wash, collect a sample to measure pH offline. Perform pH adjustment if necessary.

5. Just before removing the wash buffer out of the bioreactor, collect another sample and read offline pH to confirm iCELLis online pH is reading correctly.

6. Set the pH control of the bioreactor to 9.5±0.2.

7. Set the temp control of the bioreactor to 25° C.

8. Add 600 ml lysis buffer containing 75 mM Tris and 150 U Benzonase®/ml and having pH 9.5±0.2, and add 2.4 ml of 0.5M MgCl2.

9. Wait until the pH drops and stabilizes.

10. Take an offline pH measure and adjust iCELLis offset if necessary:

Turn on pH control (set point 9.5±0.2), and increase linear flow rate to 1.25 cm/sec and let run for 4.0 hr at 25° C.

11. Lysis time starts when the bioreactor indicates that pH reaches 9.5. Take another offline pH reading to verify it's reading correctly.

12. 2 hr after lysis starts, take sample.

13. Drain the bioreactor 4 hr after lysis starts.

14. Measure the pH and adjust if necessary (use 1M HCL if pH is too high).

Record if adjusted.

Clarification

1. Set up a filter clarification system as depicted in FIG. 10.

2. Prime the autoclaved pre-assembled Sartopure PP3 (5.0 um/1.2 um) filters by flushing the filters with 750 mL priming buffer (75 mM Tris, pH 9, 2 mM MgCl2) by using a peristaltic pump at 250 ml/min:

(a) Open the air ports on both filters and flush until buffer comes out and then close off until fully primed;

(b) Pump until all priming buffer is gone and open the air ports again (no more liquid should come out).

3. Filter the entire lysate by pumping the lysate through the filter using a peristaltic pump at 250 ml/min.

(c) Begin to slowly pump in the viral lysate until liquid comes out of the side air ports and then cap off and wipe with ethanol;

(d) Pump all of the lysate (˜600 ml) through the filters at a rate of 250 ml/min.

(e) At the end, pump through until only air comes out of the air ports (open);

(f) Separate this vessel and collect sample.

4. Flush the filter with an equal volume of flush buffer (75 mM Tris, pH 9, 2 mM MgCl2) at 250 ml/min into a separate container, and sample the flush buffer.

5. Pool the filtered lysate and flush buffer, collect another sample.

6. Immediately adjust pH of the pooled lysate of step 4 to 9 using a small amount of 1M HCl.

7. Store the pH9.0 pooled filtrate at 2-8° C. (for about 8 hours, about 10 hours, about 12 hours, about 14 hours, about 16 hours, about 18 hours, about 20 hours, about 22 hours, about 24 hours, about 26 hours, about 28 hours, about 30 hours, about 36 hours, about 40 hours, about 45 hours, about 50 hours, about 70 hours, or about 72 hours): if store stored over extended period time, such as for about 36 hours, take 6×250 μl samples before preceding to the next step.

8. Further lysis to produce depth filtrate: add 150 U/ml of Benzonase® (720 ul) plus enough 2M NaCl (90 mL) to reach 150 mM NaCl and pH 9.0, and incubate at room temperature overnight 18±4 hrs.

TFF and Diafiltration

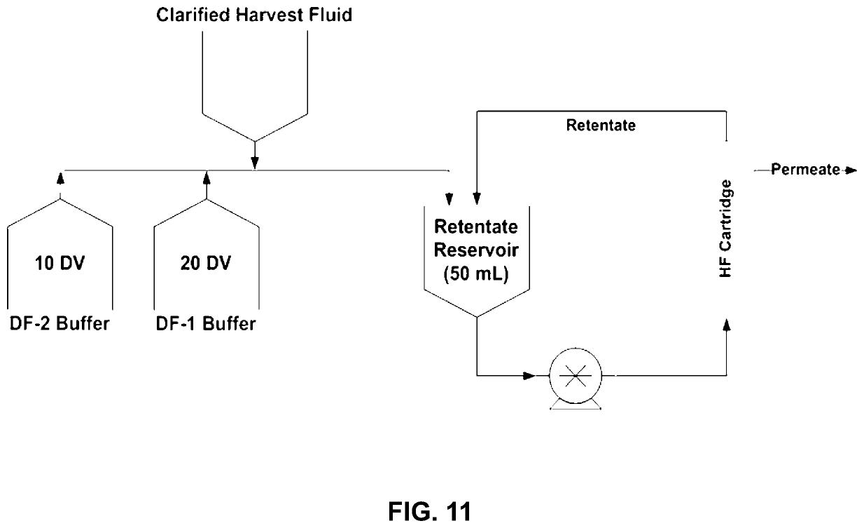

1. Set up a sterile 0.1 μm hollow fiber filter (GE) RTP HF cartridge TFF system, as depicted in FIG. 11.

2. Pre-flush the TFF system with 1 L pre-flush buffer (75 mM Tris, 2 mM MgCl2, 150 mM NaCl, pH 9.00).

3. TFF-1 Concentration:

(a) Fill retentate reservoir with ˜45 mL of filtered lysate

(b) Start pump at ˜100 mL/min to fill tubing with permeate valve closed.

(c) Once retentate reservoir reaches approximately 5 mL, add further 40 mL volume of filtered lysate to reservoir. Increase feed flow rate to prescribed flow-rate at 1.472 L/min and recirculate with permeate line closed to 5-10 mins.

(d) Post membrane conditioning open permeate valve slowly to prevent spike in flux rate to >15 LMH

(e) Concentrate the filtered lysate to 1/20 of its original volume (20× concentration), control the permeate flux at a target of 15 LMH by applying a control valve on the permeate line; the temperature is not controlled

4. TFF-1 Diafiltration 1: conduct the first diafiltration with 20 DV of the buffer 75 mM Tris, 2 mM MgCl2, 150 mM NaCl, pH 9.00; control the permeate flux at 20 Liter/m2/h (LMH); shear rate at 3000-5000 s-1; no temperature control.

5. TFF-1 Diafiltration 2: conduct the second diafiltration with 10 DV of the buffer 20 mM Tris, 8.5% w/v sucrose, pH 7.80; control the permeate flux at 30 LMH; shear rate at 3000-5000 s-1; no TMP control. After diafiltration, recover the retenate from the TFF system (tubing and the HF cartridge) to the retentate reservoir, lower the pump flow rate during product recovery.

Final Filtration

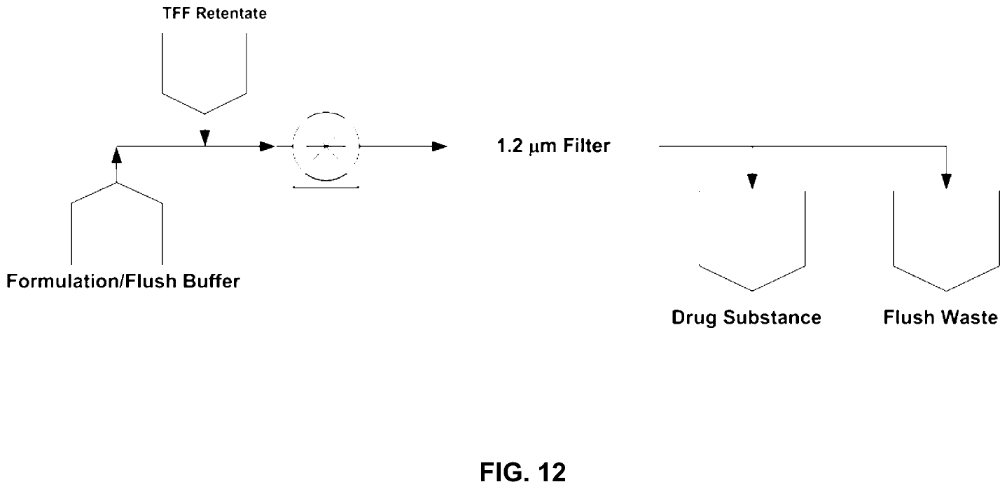

Pump TFF retentate through 1.2 um filter to Drug Substance bottle at 150 LMH pump flow rate, as depicted in FIG. 12.

### Example 6. Virus Production (I)

This example describes an example of virus production according to an embodiment of the present disclosure and the exemplary yields of an exemplary vaccinia virus from the manufacturing process.

Host Hela cells were infected with an exemplary vaccinia virus at m.o.i. of 0.02, and cultured in a microcarrier fixed-bed based bioreactor of 4 m² surface area. At 72 hours post-infection, the viruses were harvested, and subject to clarification, benzonase treatment, TFF for 20 times concentration, DF-1 and DF-2, and then the resultant purified virus preparation was kept at 2-8° C. overnight followed by final filtering. Table 1 shows measurements of the virus titer, amount of host cell protein (HCP), host cell DNA (hcDNA), and BSA in the virus preparation obtained at the end of each manufacture step.

TABLE 1
Measurements of Productivity and Purity

| | | | | HCP | hcDNA | BSA |
|---|---|---|---|---|---|---|
| Process | | Total | Overall | (μg/1 × 10⁹ | (ng/1 × 10⁹ | (ng/1 × 10⁹ |
| step | PFU/mL | PFU | Recovery | PFU) | PFU) | PFU) |
| Harvest | 7.16 × 10⁸ | 4.37 × 10¹¹ | 100% | — | 9,553 | 3,809 |
| Post-clarification | 3.48 × 10⁸ | 4.33 × 10¹¹ | 99% | 170.5 | 2,983 | 3,415 |
| Post-Benzonase ® | 2.26 × 10⁸ | 2.88 × 10¹¹ | 66% | 299.7 | 243 | 5,043 |
| Post-20X conc. | 4.70 × 10⁹ | 2.96 × 10¹¹ | 68% | 23.1 | — | 636 |
| Post-DF-1 | 4.56 × 10⁹ | 2.87 × 10¹¹ | 66% | 1.2 | — | 10 |
| Post DF-2 | 4.52 × 10⁹ | 2.63 × 10¹¹ | 60% | 0.9 | 97 | 9 |
| Post-Overnight | 4.02 × 10⁹ | 2.24 × 10¹¹ | 51% | — | — | — |
| (at 2-8° C.) | | | | | | |
| Post final filter | 3.30 × 10⁹ | 2.22 × 10¹¹ | 52% | 1.0 | 93 | 12 |

### Example 7. Virus Production (II)

This example describes an example of virus production according to an embodiment of the present disclosure and the exemplary yields of an exemplary vaccinia virus from the manufacture process.

Host Hela cells were infected with an exemplary vaccinia virus at m.o.i. of 0.001, and cultured in a microcarrier fixed-bed based bioreactor of 4 m² surface area. At 72 hours post-infection, the viruses were harvested, and subject to clarification, Benzonase® treatment, TFF for 20 times concentration, DF-1 and DF-2, and then the resultant purified virus preparation was kept at 2-8° C. overnight followed by final filtering. Table 2 shows measurements of the virus titer, amount of host cell protein (HCP), host cell DNA (hcDNA), and BSA in the virus preparation obtained at the end of each manufacture step.

TABLE 2
Measurements of Productivity and Purity

| | | | | HCP | hcDNA | BSA |
|---|---|---|---|---|---|---|
| Process | | Total | Overall | (μg/1 × 10⁹ | (ng/1 × 10⁹ | (ng/1 × 10⁹ |
| step | PFU/mL | PFU | recovery | PFU) | PFU) | PFU) |
| Harvest | 1.80 × 10⁹ | 1.09 × 10¹² | 100% | — | 633 | 1571 |
| Post-Benzonase ® | 5.71 × 10⁸ | 6.83 × 10¹¹ | 66% | 192.0 | 35 | 2081 |
| Post DF-2 | 7.57 × 10⁹ | 5.60 × 10¹¹ | 60% | 1.0 | 10 | 6 |
| Post-Overnight | 9.29 × 10⁹ | 6.83 × 10¹¹ | 51% | — | — | |
| (at 2-8° C.) | | | | | | |
| Post final filter | 6.14 × 10⁹ | 6.66 × 10¹¹ | 52% | 0.7 | 7 | 5 |

### Example 8. Virus Harvesting Comparison

This example describes an example of virus production according to an embodiment of the present disclosure and the exemplary yields of an exemplary vaccinia virus from the manufacturing process as compared to a manufacturing using a conventional hypotonic harvesting method.

In each of a series of experiments, two batches of Hela cells were infected with an exemplary modified vaccinia virus at MOI of 0.02 and grown for 48 hours in a microcarrier fixed-bed based bioreactor. One batch of the virus-infected Hela cells were harvested using a hypotonic lysis buffer, and the other batch were harvested using an exemplary high pH buffer according to the present disclosure. Table 3 below summarizes the average yield at the harvesting step.

TABLE 3
Virus yield of different harvesting methods

| Infectious Virus | Harvest | Harvest | |
|---|---|---|---|
| Titer/Surface Area | with | with | Fold |
| of Bioreactor | Hypotonic | High pH | Improvement |
| (measured after | Lysis | Lysis | in high pH |
| Harvest) | Buffer | Buffer | buffer |
| Average PFU/cm² | 2 × 10⁶ | 1.25 × 10⁷ | 625% |

While preferred embodiments of the present disclosure have been shown and described herein, such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. A method of manufacture, comprising: (i) growing a culture comprising a plurality of virus-infected host cells in a bioreactor; and (ii) harvesting from said culture a population of recombinant oncolytic virus, wherein said harvesting comprises lysing said culture to produce a lysate comprising said population of recombinant oncolytic virus, and (iii) clarifying said lysate to produce a population of purified recombinant oncolytic virus, wherein said population of purified recombinant oncolytic virus comprises at least about 50% to about 90% of said population of recombinant oncolytic virus in said lysate, and wherein said bioreactor comprises a surface area of about 0.2 m² to about 500 m².

2. The method of claim 1, wherein said bioreactor comprises a fixed bed.

3. The method of claim 2, wherein said fixed bed comprises microcarriers.

4. The method of claim 2, wherein said fixed bed comprises a volume of about 0.01 L to about 25 L.

5. The method of claim 2, wherein said fixed bed comprises a compaction density of at least about 80 g/L to about 144 g/L.

6. The method of claim 1, wherein said plurality of virus-infected host cells are seeded at a density of at least about 1,000 cells/cm² to about 150,000 cells/cm² in said bioreactor.

7. The method of claim 1, wherein said culture comprising said virus-infected host cells is grown for about 24 hours to about 96 hours.

8. The method of claim 1, wherein said culture comprising said virus-infected host cells comprises a dissolved oxygen tension (DOT) level of at least about 20% to about 100%.

9. The method of claim 1, wherein said culture comprising said virus-infected host cells is maintained at a temperature of about 32° C. to about 38° C.

10. The method of claim 1, wherein said culture comprising said virus-infected host cells is maintained at a pH of about 7.0 to about 7.5.

11. The method of claim 1, wherein said plurality of virus-infected cells have been infected with a virus at a multiplicity of infection (m.o.i.) of about 0.0005 to about 0.2.

12. The method of claim 1, wherein said harvesting comprises lysing said plurality of virus infected host cells by incubating in an alkaline buffer.

13. The method of claim 1, wherein said harvesting comprises lysing said plurality of virus infected host cells by incubating with an enzyme to degrade cell debris.

14. The method of claim 12, wherein a concentration of said enzyme, during said incubating, is from about 50 IU/mL to about 200 IU/mL.

15. The method of claim 11, wherein said incubating is for about 2 hours to about 8 hours.

16. The method of claim 11, wherein said incubating is at a temperature of about 22° C. to about 28° C.

17. The method of claim 12, wherein said enzyme to degrade cell debris comprises benzonase.

18. The method of claim 11, wherein said alkaline buffer comprises a Tris buffer.

19. The method of claim 1, wherein said host cell is selected from a group consisting of a HeLa cell, 293 cells, and Vero cells.

20. The method of claim 1, wherein said recombinant oncolytic virus comprises a recombinant oncolytic vaccinia virus.
